SEQUENCE LISTING (4 sequences)

SEQ ID NO: 1 - DNA, 1152 bp, Homo sapiens
atgctgtcccgaaagaaaaccaaaaacgaagtgtccaagccggccgaggtgcaggggaagtacgtgaagaaggagacgtcgcctctgcttcggaatcttatgccttcattcatccggcatggtccaacaattccaagacgaactgatatctgtcttccagattcaagccctaatgccttttcaacttctggagatgtagtttcaagaaaccagagtttccttagaactccaattcaaagaacacctcatgaaataatgagaagagaaagcaacagattatctgcaccttcttatcttgccagaagtctagcagatgtccctagagagtatggttcttctcagtcatttgtaacggaagttagttttgctgttgaaaatggagactctggttcccgatattattattcagacaatttttttgatggtcagagaaagcggccacttggagatcgtgcacatgaagactacagatattatgaatacaaccatgatctcttccaaagaatgccacagaatcaggggaggcatgcttcaggtattgggagagttgctgctacatctttaggaaatttgactaaccatggttctgaagatttaccccttcctcctggctggtctgtggactggacaatgagagggagaaaatattatatagatcataacacaaatacaactcactggagccatcctcttgagcgagaaggacttcctcctggatgggaacgagttgagtcatccgaatttggaacctattatgtagatcacacaaataagaaggcccaatacaggcatccctgtgctcctagtgtacctcggtatgatcaaccacctcctgtcacataccagccacagcaaactgaaagaaatcagtcccttctggtacctgcaaatccatatcatactgcagaaattcctgactggcttcaggtttacgcacgagcccctgtgaaatatgaccacattctgaagtgggaactcttccagctggctgacctggatacataccagggaatgctaaagttgctcttcatgaaagaattggagcagattgttaaaatgtatgaagcatacagacaagcccttcttacagagttggaaaaccgaaagcagagacagcagtggtatgcccaacaacatggaaaaaatttttga

SEQ ID NO: 2 - DNA, 21 bp, Homo sapiens
aagtacgtgaagaaggagacg

SEQ ID NO: 3 - DNA, 21 bp, Homo sapiens
aagatttaccccttcctcctg

SEQ ID NO: 4 - DNA, 21 bp, Homo sapiens
aattcctgactggcttcaggt

(19) United States
(12) Patent Application Publication
(10) Pub. No.: US 2024/0026354 A1
(43) Pub. Date: Jan. 25, 2024

(54) SUPPRESSING HIPPO SIGNALING IN THE STEM CELL NICHE PROMOTES SKELETAL MUSCLE REGENERATION

(71) Applicants: Baylor College of Medicine, Houston, TX (US); Texas Heart Institute, Houston, TX (US)

(72) Inventors: Richard A.F. Dixon, Houston, TX (US); Qi Liu, Houston, TX (US); James T. Willerson, Houston, TX (US); James F. Martin, Pearland, TX (US)

(21) Appl. No.: 18/037,029

(22) PCT Filed: Nov. 18, 2021
(86) PCT No.: PCT/US2021/072500

§ 371 Date: May 15, 2023

Related U.S. Application Data
(60) Provisional application No. 63/116,754, filed on Nov. 20, 2020.

(51) Int. Cl. C12N 15/113; C12N 15/86; A61P 21/00

(52) U.S. Cl. CPC C12N 15/113; C12N 15/86; A61P 21/00; C12N 2310/122; C12N 2750/14143

(57) ABSTRACT

Embodiments of the disclosure include methods for generating skeletal muscle by targeting the Hippo pathway. In particular embodiments, an individual with a need for skeletal muscle generation is provided an effective amount of a shRNA molecule that targets the SAV1 gene. Particular shRNA sequences are disclosed.

### CROSS-REFERENCE TO RELATED APPLICATIONS

This application claims the benefit of priority to U.S. Provisional Patent Application Ser. No. 63/116,754, filed Nov. 20, 2020, hereby incorporated by reference in its entirety.

### STATEMENT REGARDING FEDERALLY SPONSORED RESEARCH OR DEVELOPMENT

This disclosure was made with government support under NIH R01 HL 127717 awarded by National Institutes of Health (NIH). The government has certain rights in the disclosure.

### TECHNICAL FIELD

The present disclosure concerns at least the fields of cell biology, molecular biology, and medicine.

### BACKGROUND

Although the incidence of lower extremity ischemia is increasing in older adults, current treatment options, such as endovascular surgery and gene and stem cell-based therapies, are ineffective in restoring limb viability.¹⁻³ These current strategies focus on revascularization and neovascularization to re-establish perfusion; however, regeneration of skeletal muscle to replace damaged muscles and restore muscular function is seldom addressed.⁴ Skeletal muscle makes up the primary mass of the limb and contains most of the vessels involved in limb circulation. As such, skeletal muscle is vulnerable to vascular damage, and injury to the muscle correlates directly with the severity and duration of ischemia.^{5,6} Thus, muscle regeneration in conjunction with perfusion restoration is crucial to successfully treating lower extremity ischemia.

The gradual age-related decline in the regenerative ability of skeletal muscle is a fundamental challenge in skeletal muscle repair.^{7,8} One approach to promote muscular regeneration of adult stem cells is to target local environmental constraints. The regeneration of adult functional skeletal muscle relies on the continued production of myoblasts from activated satellite cells (SCs), which are normally quiescent skeletal muscle stem cells in adult muscles.^{9,10} In response to injury (e.g., during ischemia or muscle damage or injury), the SCs become activated and start proliferating to self-renew and differentiate into skeletal muscle cells.¹¹ SCs, which express myogenic—determinant PAX7 (a member of the paired box transcription factor family), are protected by the myofibers in a unique anatomical niche (microenvironment) between the myofiber plasma membrane and the basal lamina and are located in close proximity to endothelial cells (ECs).¹² The dynamic interactions between SCs and their niche components (e.g., myofibers, vascular cells, extracellular matrix, and diffusible factors) govern the quiescence, activation, proliferation, and differentiation of SCs.¹³ Myofibers communicate with SCs by secreting signaling molecules that bind to receptors on SCs, directing their fate.¹⁴ The physiological state of the myofiber or its intrinsic signaling pathways can be modulated to affect SC activation and expansion, which in turn, can activate nearby ECs by paracrine and autocrine signaling to promote angiogenesis.¹⁵

Hippo signaling, a highly conserved kinase cascade pathway, inhibits cell proliferation by modulating the signaling transcriptome associated with tissue growth.¹⁶ The mammalian core Hippo-signaling components include the Ste20 kinases Mst1 and Mst2 that are orthologous to the Drosophila Hippo kinase. Mst kinases, when complexed with the Salvador (SAV1 or SAV) scaffold protein, phosphorylate the Large Tumor Suppressor Homolog (Lats) kinases. Mammalian Lats 1 and Lats2 are NDR family kinases and are orthologous to Drosophila Warts. Lats kinases, in turn, phosphorylate Yap and Taz, two related transcriptional co-activators that are the most downstream Hippo-signaling components and partner with transcription factors such as Tead to regulate gene expression. Yap also interacts with β-catenin, an effector of canonical Wnt signaling to regulate gene expression. Upon phosphorylation, Yap and Taz are excluded from the nucleus and rendered transcriptionally inactive. The disruption of the Hippo pathway has been found to promote tissue regeneration.¹⁷

The age-associated progressive decline in skeletal muscle regeneration limits

the ability to treat skeletal muscle injury. The regeneration of skeletal muscles depends on the activity of the resident stem cells that are protected by myofibers in a unique anatomical niche and that reside adjacent to the endothelial cells. Inhibiting the Hippo pathway in myogenic cells could alter the composition of the myofibers' secretome and enrich the local milieu with factors that will stimulate and promote the proliferation and differentiation (myogenesis) of SCs and angiogenesis in adjacent ECs, along with enhancing neovascularization. This strategy may overcome the current hurdle of functional skeletal muscle regeneration in aged populations.

### BRIEF SUMMARY

The present disclosure concerns methods and compositions that address a long-felt need in the art to activate signals for SC proliferation and self-renewal in the muscle stem cell niche by manipulating the Hippo pathway in myofibers to promote myogenesis simultaneously with angiogenesis and neovascularization to provide therapy for skeletal muscle conditions.

The disclosure provides, for example, methods of increasing angiogenesis and/or myogenesis in skeletal muscle, of regenerating myofibers in skeletal muscle, and of inducing proliferation and, in some embodiments, differentiation, of satellite cells in skeletal muscle. The methods comprise delivering to skeletal muscle cells, including satellite cells, an effective amount of a composition comprising at least one inhibitory nucleic acid, wherein the inhibitory nucleic acid targets Salvador (SAV1).

In some embodiments, the composition is administered to a mammalian subject in need thereof. For example, skeletal muscle of the mammal can be ischemic or atrophic. In some embodiments, the skeletal muscle has suffered traumatic injury. In certain embodiments, the subject has a condition selected from the group consisting of limb ischemia, peripheral vascular disease, and sarcopenia.

The methods of the disclosure can also be in vitro or ex vivo methods.

The disclosure further provides a method of treating limb ischemia in a mammalian subject, the method comprising delivering to skeletal muscle cells of an ischemic limb in the subject an effective amount of a composition comprising at least one inhibitory nucleic acid, wherein the inhibitory nucleic acid targets SAV1.

Also provided are inhibitory nucleic acids targeting SAV1 for use in the methods of the disclosure.

In some embodiments, the inhibitory nucleic acid has, or is encoded by a

sequence having, at least 80% identity to a nucleotide sequence selected from the group consisting of SEQ ID NO: 2, SEQ ID NO: 3, and SEQ ID NO: 4.

In a specific embodiment, the composition comprises (i) an inhibitory nucleic acid having, or encoded by a sequence having, at least 80% identity to SEQ ID NO: 2, (ii) an inhibitory nucleic acid having, or encoded by a sequence having, at least 80% identity to SEQ ID NO: 3, and (iii) an inhibitory nucleic acid having, or encoded by a sequence having, at least 80% identity to SEQ ID NO: 4.

In certain embodiments, the inhibitory nucleic acid has, or is encoded by a sequence having, at least 85%, 90%, 95%, 96%, 97%, 98%, or 99% identity to a sequence selected from the group consisting of SEQ ID NO: 2, SEQ ID NO: 3, and SEQ ID NO: 4. In particular embodiments, the inhibitory nucleic acid has or is encoded by a sequence selected from the group consisting of SEQ ID NO: 2, SEQ ID NO: 3, and SEQ ID NO: 4.

In one embodiment, the composition comprises a nucleic acid construct comprising: (i) a nucleic acid having the nucleotide sequence set forth in SEQ ID NO: 2, (ii) a nucleic acid having the nucleotide sequence set forth in SEQ ID NO: 3, and (iii) a nucleic acid having the nucleotide sequence set forth in SEQ ID NO: 4; wherein nucleic acids (i)-(iii) are operably linked to a promoter.

The inhibitory nucleic acid can be, for example, an inhibitory DNA or RNA molecule, such as an antisense DNA oligonucleotide, an antisense RNA oligonucleotide, or a short hairpin RNA, a short interfering RNA. In one embodiment, the shRNA is at least 43 nucleotides in length. In one embodiment, the shRNA is less than 138 nucleotides in length. In certain embodiments, the shRNA comprises a loop structure of between 5 and 19 nucleotides in length, preferably between 4 and 10 nucleotides in length.

In some embodiments, a nucleotide sequence encoding an inhibitory RNA is comprised in a nucleic acid construct. In such embodiments, the inhibitory RNA is preferably expressed in skeletal muscle cells. Accordingly, the nucleotide sequence encoding the inhibitory RNA can be operably linked to a promoter, such as a tissue-specific promoter. In a particular embodiment, the promoter is a cardiac troponin T promoter. In some embodiments, the nucleic acid construct comprises at least one post-transcriptional regulatory element, for example, a woodchuck post-transcriptional regulatory element. In some embodiment, the nucleic acid construct comprises sequences encoding a 3′ microRNA-30 sequence and a 5′ microRNA-30 sequence. In certain embodiments, the nucleic acid construct comprises 5′ and 3′ inverted terminal repeats.

In methods utilizing more than one inhibitory RNA, the sequences encoding each inhibitory RNA can be on the same nucleic acid construct or on different nucleic acid constructs. Transcription of each inhibitory RNA sequence can be controlled by its own promoter, or a single promoter can control transcription of more than one inhibitory RNA sequence. In one embodiment, nucleotide sequences encoding multiple inhibitory RNAs are regulated by a single promoter.

In some embodiments of the disclosure, an inhibitory nucleic acid targeting SAV1 or a nucleotide sequence encoding an inhibitory nucleic acid targeting SAV1 is comprised in a vector. The vector can be a viral vector or a non-viral vector. Viral vectors can be derived, for example, from adeno-associated virus (AAV) or from lentivirus. In some embodiments, the vector is a non-integrating vector, i.e., it does not integrate into the target cell genome.

It is specifically contemplated that any limitation discussed with respect to one embodiment of the disclosure may apply to any other embodiment of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Brief Summary, Detailed Description, Claims, and

### BRIEF DESCRIPTION OF DRAWINGS

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings, in which:

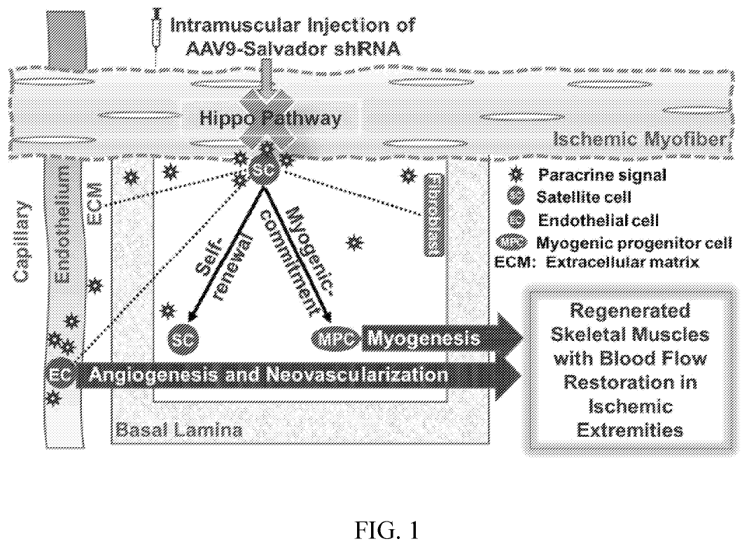

FIG. 1 illustrates the AAV9 Salvador shRNA strategy of the present disclosure, which recreates a myofiber-guided regenerative stem cell niche and promotes simultaneous myogenesis and angiogenesis-neovascularization for the functional recovery of skeletal muscles in ischemic extremities. These myofibers release paracrine elements to activate (i) satellite cells to proliferate and self-renew for myogenesis and (ii) endothelial cells to stimulate angiogenesis and neovascularization.

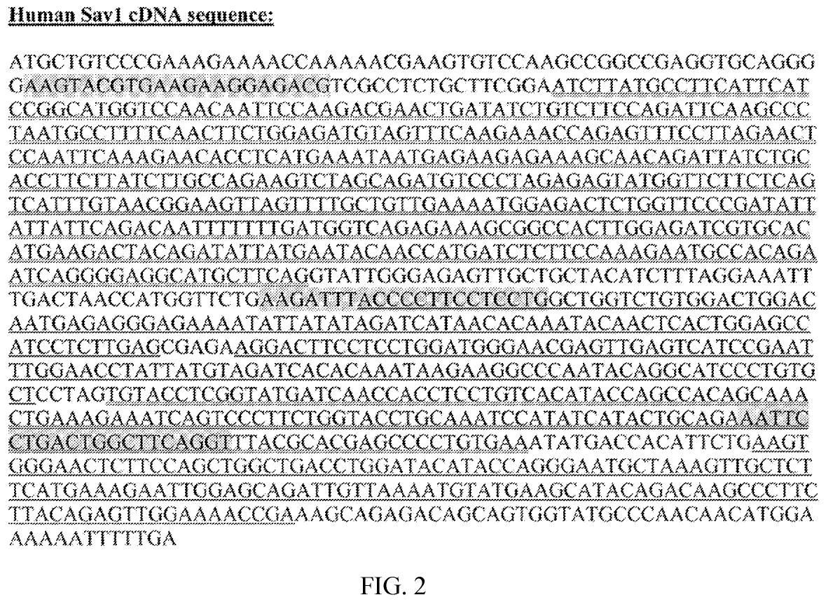

FIG. 2 shows the cDNA sequence of human SAV1 (SEQ ID NO: 1). The shaded regions indicate examples of target sequences for inhibitory nucleic acids. Alternating exons are indicated by the presence or absence of double underlining. Protein structural domains are indicated by single underlined sequences, in 5′ to 3′ order: WW domain, WW domain, SARAH domain.

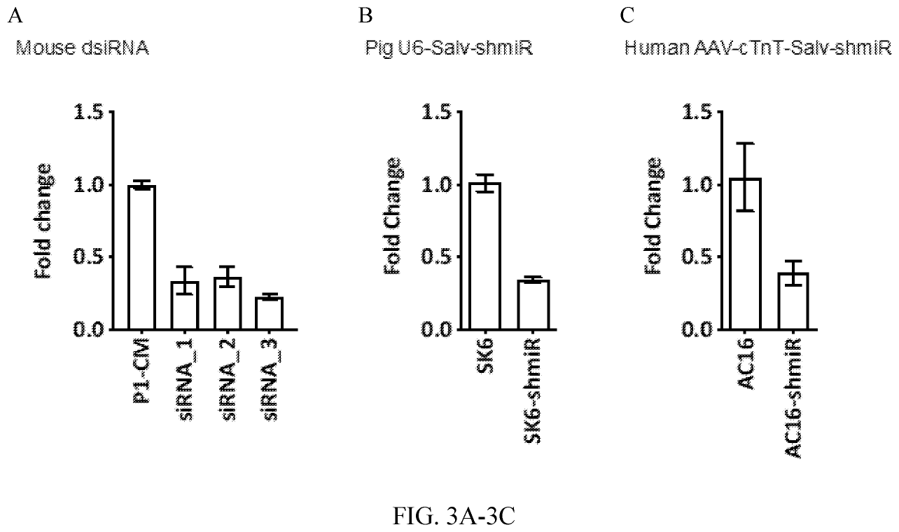

FIG. 3A-3C show that siRNA or shRNA targeting SAV1 effectively reduces SAV1 mRNA expression. Inhibitory RNA corresponding to SEQ ID NO: 2, SEQ ID NO: 3, or SEQ ID NO: 4 was transfected into neonatal cardiomyocytes. SAV1 mRNA levels were measured using quantitative RT-PCR. All three siRNAs effectively reduced SAV1 mRNA levels.

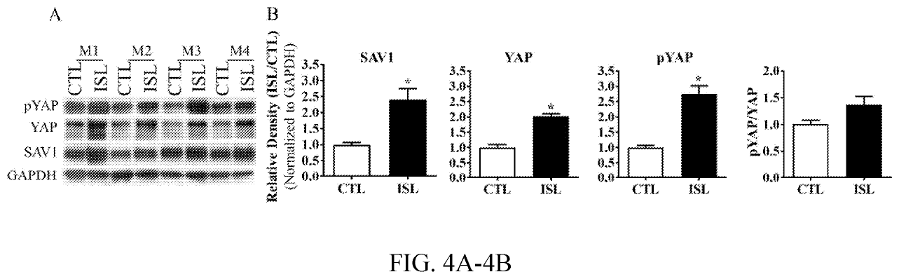

FIG. 4A-4B show that hindlimb ischemia upregulated Hippo pathway core

proteins. FIG. 4A is a Western blot showing Salvador (SAV1), total YAP and phosphorylated YAP (pYAP) levels in gastrocnemius muscles of contralateral nonischemic legs (CTL) and ischemic legs (ISL) at 14 days after induction of unilateral hindlimb ischemia. GAPDH was used for loading control. FIG. 4B is a semi-quantitative analysis showing SAV1, total YAP and pYAP upregulation in gastrocnemius muscles of ISL as compared to CTL. The ratio of pYAP to total YAP was not changed. *P<0.05 by Mann Whitney test; n=4 mice. M1-M4: Mouse #1 to mouse #4. Data are mean±SEM.

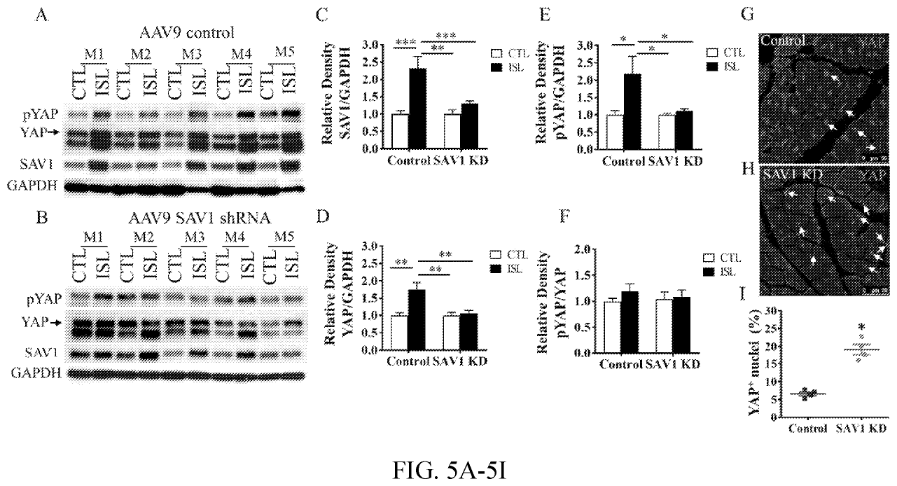

FIG. 5A-5I show that AAV9 SAV1 shRNA downregulated SAV1, YAP, and pYAP expression. FIGS. 5A and 5B are Western blots showing SAV1, total YAP, and pYAP protein levels in gastrocnemius muscles of contralateral nonischemic legs (CTL) and ischemic legs (ISL) 7 days after the intramuscular injection of AAV9 control and AAV9 SAV1 shRNA into ISL. FIG. 5C-5F show semiquantitative analysis showing reduced SAV1, total YAP, and pYAP expression in SAV1 shRNA-treated ISL (SAV1 KD) as compared to control-treated ISL (Control). There was no significant change in the pYAP/YAP ratio. *P<0.05, **P<0.01, ***P<0.001, n=5 mice/group by one-way ANOVA with Dunnett's multiple comparison test. FIGS. 5G and 5H, are representative immunofluorescence images showing more extensive myogenic YAP nuclear localization (white arrows) after SAV1 knockdown (KD) than after control treatment 14 days after AAV9 administration. FIG. 5I shows quantification of YAP nuclear localization in myofibers. *P<0.05 by nonparametric Mann Whitney test, n=4 mice/group. Data are mean±SEM.

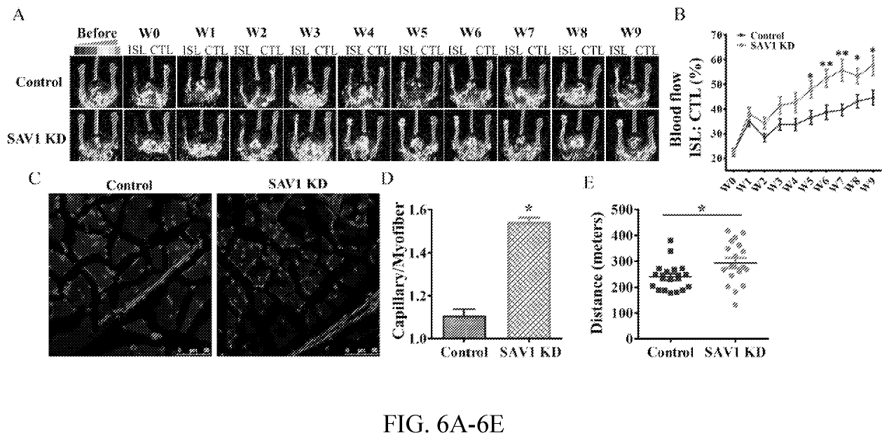

FIG. 6A-6E show functional outcome of hindlimb ischemia after AAV9 shRNA-mediated SAV1 knockdown in ischemic legs. FIG. 6A shows representative laser Doppler perfusion images showing the time-course study on the restoration of blood flow to ischemic limbs in AAV9 SAV1 shRNA-treated mice (SAV1 KD) as compared to AAV9 control-treated mice (control). FIG. 6B shows that SAV1 KD resulted in significantly improved perfusion as compared to control-treated mice by weeks 5-9 (n=18 mice/group). FIG. 6C shows that more red fluorescent lectin-stained capillaries and arterioles were observed in the gastrocnemius muscles of SAV1 KD mice than in those of control mice. FIG. 6D shows quantification of lectin⁺ capillaries per myofiber (n=4 mice/group). FIG. 6E shows that SAV1 KD resulted in significantly greater treadmill endurance by week 7 (n=18 mice/group). Data are mean±SEM. *P<0.05, **P<0.01 by nonparametric Mann Whitney test.

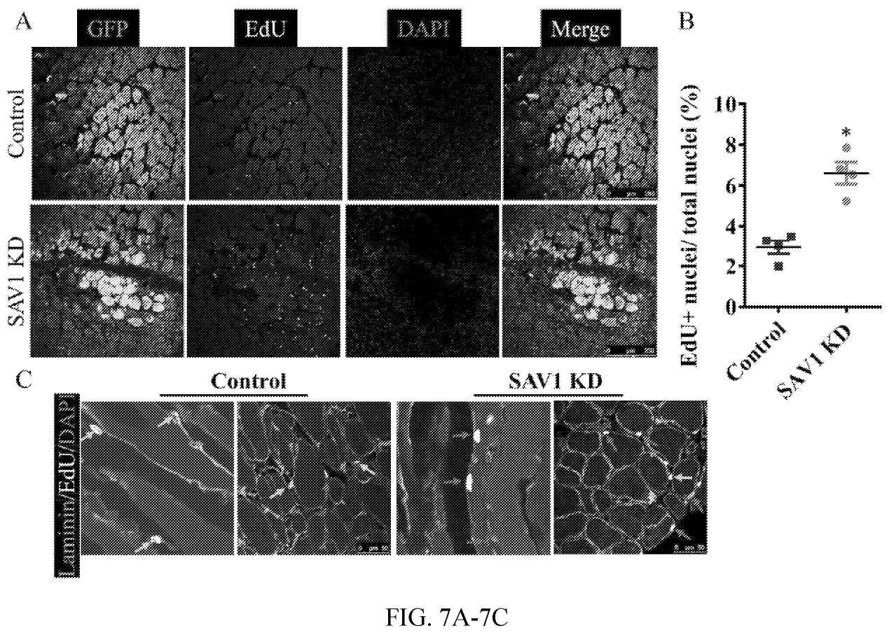

FIG. 7A-7C show that AAV9 shRNA-mediated SAV1 knockdown in ischemic legs promoted cell proliferation. FIG. 7A shows representative immunofluorescence images illustrating more EdU⁺ cells after SAV1 knockdown in ischemic gastrocnemius muscles than after control treatment. DAPI counter staining of nuclei was performed. White immunofluorescent pseudo-color was applied to EdU incorporated nuclei. FIG. 7B shows that the average ratio of EdU positive nuclei to total nuclei was significantly higher in ischemic gastrocnemius muscles with SAV1 knockdown than in those with control treatment. n=4 mice/group. *P<0.05 by nonparametric Mann Whitney test. FIG. 7C shows representative immunofluorescence images of gastrocnemius muscle sections showing the distribution of EdU nuclei around myofibers in the control and SAV1 knockdown groups. Laminin staining was used to identify the basal lamina. Yellow arrows mark EdU-labelled nuclei in the interstitium between myofibers; red arrows mark EdU-labelled nuclei along the periphery of longitudinal skeletal muscle fibers or between the myofiber membrane and basal lamina.

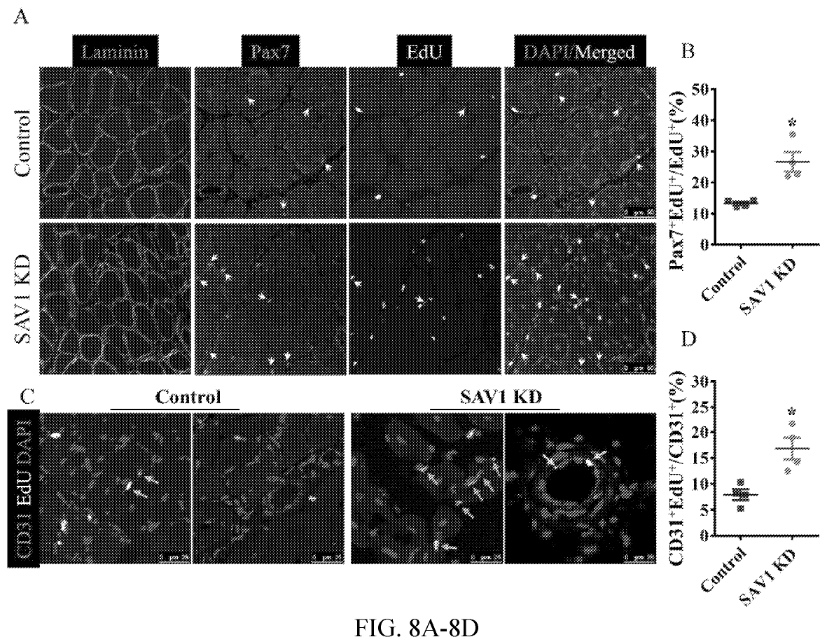

FIG. 8A-8D show that AAV9 shRNA-mediated SAV1 knockdown promoted satellite cell and endothelial cell proliferation. FIG. 8A shows representative immunofluorescence images depicting quiescent EdU⁻Pax7⁺ (white arrows) and proliferating EdU⁺Pax7⁺ satellite cells (yellow arrows). FIG. 8B shows that a significantly higher ratio of EdU⁺Pax7⁺ to total EdU nuclei (percentage) was detected in SAV1 KD group than in the control group. FIG. 8C shows proliferating CD31⁺ endothelial cell showing nuclear localization of EdU (purple arrows). CD31⁺EdU⁺ vessel sprouting (green arrows) and CD31⁺EdU⁺ collateral vessels (orange arrows) were observed in SAV1 KD group. White immunofluorescent pseudo-color was applied to EdU incorporated nuclei. FIG. 8D shows the ratio of CD31⁺EdU⁺ cells to total CD31⁺ cells (%) per high power field. n=4 mice/group, *P<0.05. Nonparametric Mann Whitney test was used for pairwise comparisons.

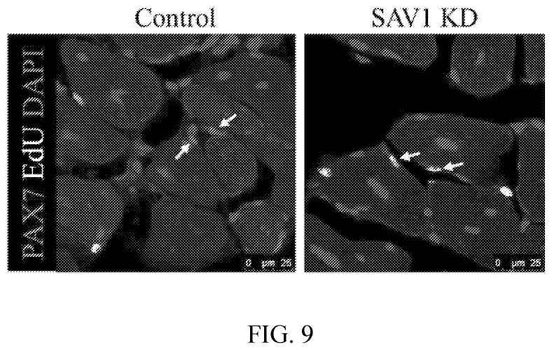

FIG. 9 shows representative immunofluorescence images showing quiescent EdU-Pax7+ (white arrows) and proliferating EdU+Pax7+ satellite cells (yellow arrows) at 14 days after AAV9 control or AAV9 SAV1 shRNA injection into mouse ischemic hindlimbs.

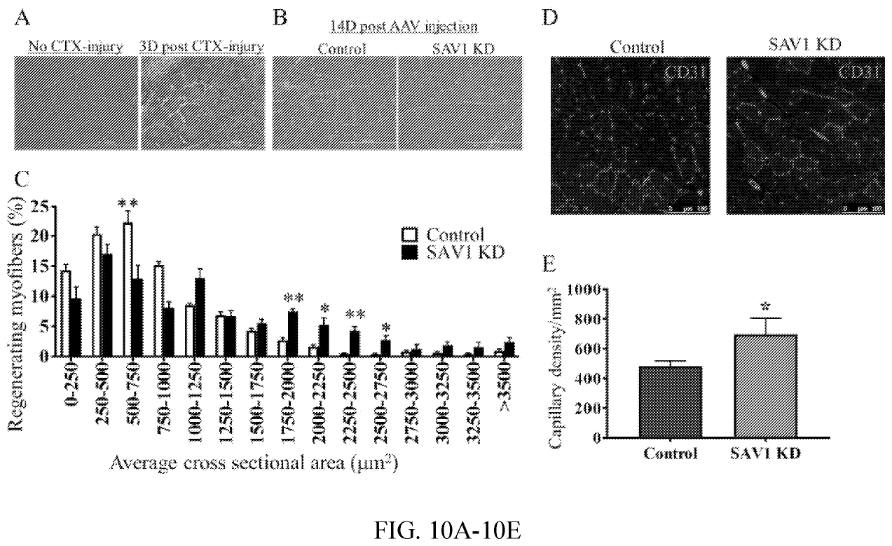

FIG. 10A-10E show that AAV9 SAV1 shRNA promoted skeletal muscle regeneration after muscle injury. FIG. 10A shows representative images of the tibialis anterior muscle showing normal muscle that did not receive cardiotoxin (CTX) injections and muscle containing inflammatory cell infiltration and myofiber necrosis at day 3 after CTX injection. FIG. 10B shows representative images of centrally nucleated myofibers at 14 days after AAV9 control (Control) and AAV9 SAV1 shRNA (SAV1 KD) treatment. FIG. 10C shows that SAV1 shRNA-treated tibialis anterior muscle had large-caliber regenerated myofibers as compared to control-treated muscle. n=3 mice/group, *P<0.05, **P<0.01. FIG. 10D shows representative immunofluorescence images of cross-sections of tibialis anterior muscles stained with CD31 to visualize capillaries and blood vessels. FIG. 10E shows that capillary density was significantly higher in the SAV1 KD group than in the control group. n=4 mice/group, *P<0.05. Nonparametric Mann Whitney test was used for pairwise comparisons.

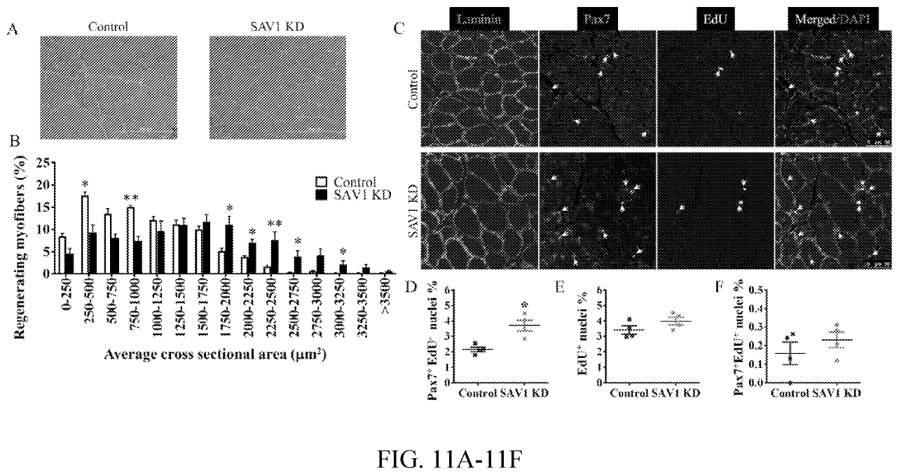

FIG. 11A-11F show that AAV9 SAV1 shRNA treatment promoted skeletal muscle regeneration after cardiotoxin-induced injury in aged mice. FIG. 11A shows representative images of hematoxylin and eosin staining showing the regenerating tibialis anterior muscle fiber cross sectional areas at 14 days after treatment with AAV9 control and AAV9 SAV1 shRNA (SAV1 KD). FIG. 11B shows a frequency distribution analysis of regenerative fiber cross sectional area, n=3/group, *P<0.05, **P<0.01. FIG. 11C shows the presence of Pax7⁺EdU⁻ cells (white arrows) and Pax7⁺EdU⁺ cells (yellow arrows). FIG. 11D shows that the ratio of Pax7⁺EdU⁻ cells was significantly higher in the SAV1 KD group than in the control group. The percentages of EdU-positive nuclei (FIG. 11E) and Pax7⁺EdU⁺ cells (FIG. 11F) were comparable at 14 days after AAV9 control and AAV9 SAV1 shRNA treatment. n=4 mice/group, *P<0.05. Nonparametric Mann Whitney test was used for pairwise comparisons.

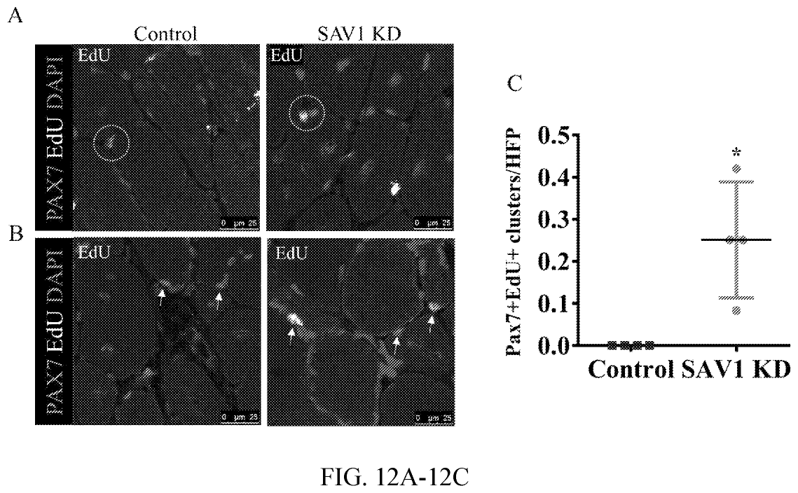

FIG. 12A-12C show the tibialis anterior muscle of aged mice stained with Pax7 antibody at 14 days after AAV9 control or AAV9 SAV1 shRNA injection following cardiotoxin induced muscle injury. FIG. 12A shows the presence of adjacent Pax7⁺EdU⁻ and Pax7⁻EdU⁺ cells (yellow circle). FIG. 12B shows EdU and Pax7 double-positive nuclei (yellow arrows) showing activated satellite cell clusters nuclei, yellow arrows) in SAV1 shRNA-treated tibialis anterior muscle; Pax7⁺EdU⁻ satellite cells (white arrows) were seen in both control and SAV1 KD groups. FIG. 12C shows the average numbers of Pax7⁺EdU⁺ SC clusters nuclei) per high power field (HPF). n=4 mice/group.

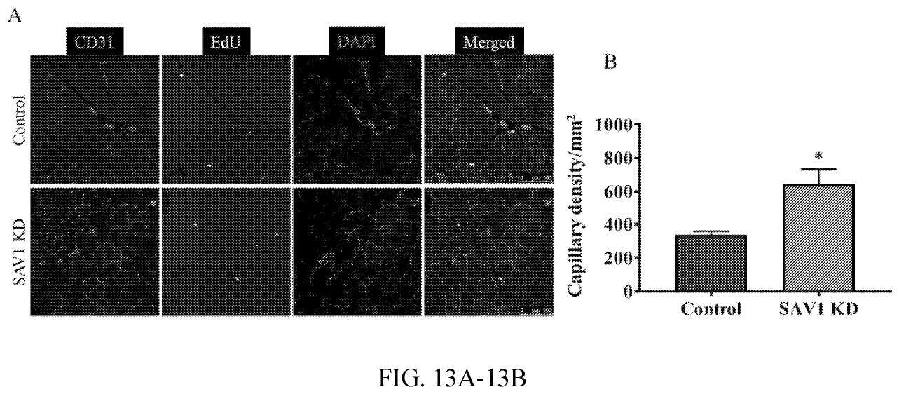

FIG. 13A-13B show that SAV1 knockdown in CTX-injured tibialis anterior muscle of aged mice promoted angiogenesis. FIG. 13A shows representative immunofluorescent images of cross-sections of tibialis anterior muscles showing CD31-positive capillaries and blood vessels. FIG. 13B shows that capillary density was significant higher in SAV1KD group than in control group. n=4 mice/group, *P<0.05. Nonparametric Mann Whitney test.

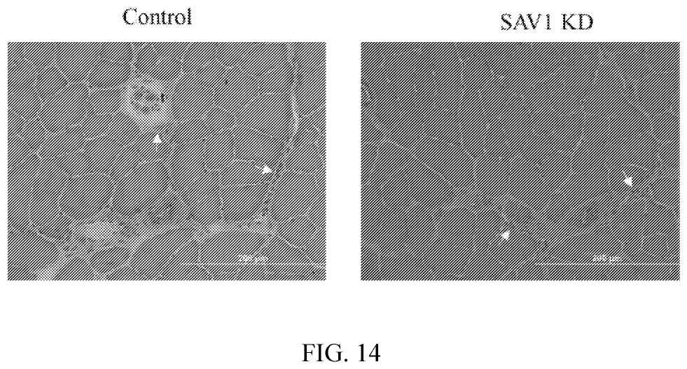

FIG. 14 shows representative images of H&E stained tibialis anterior muscle showing presence of inflammatory infiltration in the enlarged interstitial space (white arrows) and around myofibers (yellow arrows) and blood vessels (blue arrows).

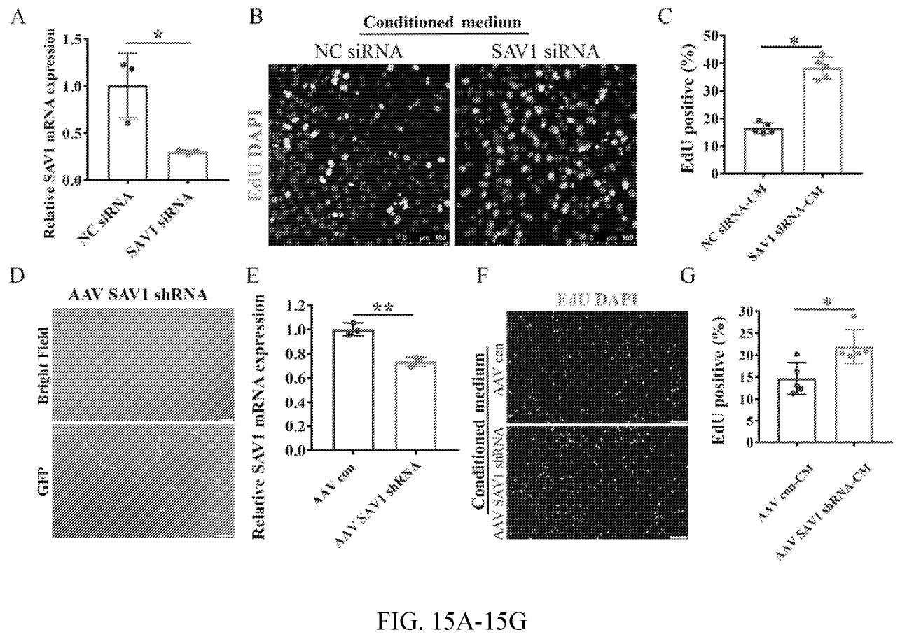

FIG. 15A-15G show that conditioned medium generated from SAV1 silencing in C2C12 myotubes promoted satellite cell proliferation in vitro. FIG. 15A shows RT-qPCR indicating that the relative expression level of SAV1 mRNA was significantly decreased after transfection of C2C12 myotubes with SAV1 siRNA, as compared to transfection with negative control (NC) siRNA for 48 hours. An unpaired t test was used for analysis; data were generated from 3 independent assays. FIG. 15B shows representative fluorescence images showing the incorporation of EdU into nuclei of satellite cells after 24-hour culture in conditioned medium collected from C2C12 myotubes after SAV1knock down (SAV1 siRNA-CM), and from C2C12 myotubes in the absence of SAV1 knock down (NC siRNA-CM). FIG. 15C shows quantification of EdU incorporation in satellite cells grown in NC siRNA-CM and SAV1 siRNA-CM. An unpaired t test was used; data were generated from 5 independent assays. FIG. shows representative phase contrast and fluorescence images of C2C12 myotubes transduced with AAV9 SAV1 shRNA at day 4. FIG. 15E shows RT-qPCR revealing the relative expression level of SAV1 mRNA analyzed 7 days after AAV control and AAV SAV1 shRNA transduction in C2C12 myotubes. An unpaired t test was used; data were generated from 3 independent assays. FIG. 15F shows representative images of EdU incorporation of satellite cells grown in conditioned medium from C2C12 myotubes containing AAV SAV1 shRNA induced SAV1 knock down (AAV SAV1 shRNA-CM), and from C2C12 myotubes in the absence of SAV1 knock down by AAV control (AAV con-CM). FIG. 15G shows that satellite cells grown in AAV SAV1 shRNA-CM exhibited a higher percentage of EdU nuclei than those grown in AAV con-CM. An unpaired t test was used; data were generated from 5 independent assays. *P<0.05. **P<0.01.

### DETAILED DESCRIPTION

### I. Exemplary Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined. The terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. The term “about” is used according to its plain and ordinary meaning in the area of cell and molecular biology to indicate that a value includes the standard deviation of error for the device or method being employed to determine the value. The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

Recitation of ranges of values herein are merely intended to serve as a shorthand method of referring individually to each separate value falling within the range, unless otherwise indicated herein, and each separate value is incorporated into the specification as if it were individually recited herein.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment.

Furthermore, the particular features, structures, or characteristics may be combined in any suitable manner in one or more embodiments. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

As used herein, the term “nucleotide sequence” or “nucleic acid” refers to a polymer of DNA or RNA having a combination of purine and pyrimidine bases, sugars, and covalent linkages between nucleosides including a phosphate group in a phosphodiester linkage. A nucleic acid can be single-stranded or double-stranded, and will optionally contain synthetic, non-natural or altered nucleotide bases capable of incorporation into DNA or RNA polymers. The term “polynucleotide” is used interchangeably with the term “oligonucleotide.” The term “nucleotide sequence” is interchangeable with “nucleic acid sequence” unless otherwise clearly stated.

In some cases, nucleic acid analogs are included that may have alternate backbones or non-natural internucleoside linkages, comprising, for example, modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like. See, for example, U.S. Pat. No. 7,410,944. Examples of modified phosphorous-containing backbones include phosphoramide, phosphorothioate, phosphorodithioate, chiral phosphorothioate, O-methylphophoroamidite phosphotriester, aminoalkylphosphotriester, alkyl phosphonate, thionoalkylphosphonate, phosphinate, phosphoramidate, thionophosphoramidate, thionoalkylphosphotriester, boranophosphate, and various salt forms thereof. Examples of the non-phosphorous containing backbones described above are known in the art, e.g., U.S. Pat. No. 5,677,439, each of which is herein incorporated by reference. Other analog nucleic acids include those with positive backbones, non-ionic backbones, and non-ribose backbones, including those described in U.S. Pat. Nos. 5,235,033 and 5,034,506. Modification of the ribose-phosphate backbone may facilitate the addition of moieties such as labels, or increase the stability and half-life of such molecules in physiological environments.

Nucleic acids can contain substituted or modified sugar moieties, e.g., 2′-O-methoxyethyl sugar moieties or carbocyclic sugars. Nucleic acids can also contain modified nucleosides (nucleoside analogs), i.e., modified purine or pyrimidine bases, e.g., 5-substituted pyrimidines, 6-azapyrimidines, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), 2-thiouridine, 4-thiouridine, 5-(carboxyhydroxy methyl)uridine, 5′-carboxymethylaminomethyl-2-thiouridine, 5-carboxymethylaminomethyluridine, 5-methoxyaminomethyl-2-thiouridine, 5-methylaminomethyluridine, 5-methylcarbonylmethyl uridine, 5-methyloxyuridine, 5-methyl-2-thiouridine, 4-acetylcytidine, 3-methylcytidine, propyne, quesosine, wybutosine, wybutoxosine, beta-D-galactosylqueosine, N-2, N-6 and O-substituted purines, inosine, 1-methyladenosine, 1-methylinosine, 2,2-dimethylguanosine, 2-methyladenosine, 2-methylguanosine, N6-methyladenosine, 7-methylguanosine, 2-methylthio-N-6-isopentenyl adenosine, beta-D-mannosylqueosine, uridine-5-oxyacetic acid, 2-thiocytidine, threonine derivatives, and the like. See, for example, U.S. Pat. No. 7,410,944.

In the context of nucleic acids, the terms “hybridize,” “bind,” “target,” or variations thereof refer to a sufficient degree of complementarity or base pairing between an complementary or inhibitory nucleic acid sequence and a target DNA or mRNA, such that a stable and specific interaction occurs between them. Specific hybridization occurs when sufficient interaction occurs between the complementary or inhibitory nucleotide sequence and its intended target nucleic acids, in the substantial absence of non-specific binding of the complementary or inhibitory nucleotide sequence to non-target sequences under predetermined conditions, preferably under biological or physiological conditions.

Preferably, specific hybridization results in inhibition, i.e., the interference with normal expression of the gene product encoded by the target DNA or mRNA. Full complementarity between the target sequence and the inhibitory RNA is not required. The inhibitory nucleotide sequence, e.g., a single-stranded antisense oligonucleotide or the antisense sequence of a double-stranded inhibitory RNA, is sufficiently complementary if it binds to the target sequence under predetermined conditions and inhibits target gene expression. For example, an antisense nucleotide sequence can be designed to specifically hybridize to the replication or transcription regulatory regions of a target gene, or the translation regulatory regions such as translation initiation region and exon/intron junctions, or the coding regions of a target mRNA.

As used herein, an “antisense” nucleic acid sequence or oligonucleotide is a sequence of DNA or RNA that can bind to via base-pairing a target “sense” sequence. In some instances, the sense sequence is a nucleic acid encoding a protein, such that the antisense sequence is complementary to the coding strand of a double-stranded cDNA molecule or complementary to an mRNA sequence. The antisense sequence can be fully or partially complementary to the sense sequence. Antisense nucleotide sequences can be designed to specifically hybridize to a particular region of a desired target protein or mRNA to interfere with replication, transcription, or translation. An antisense sequence can be complementary to any length of sense sequence, for example, to at least about 15, 16, 17, 18, 19, 20, 21, 22, 23, 24, or 25 nucleotides.

The terms “inhibitory nucleic acid” and “inhibitory oligonucleotide” are used interchangeably and refer to a molecule that knocks down expression of a target gene by preventing translation of the corresponding mRNA. As discussed above, expression is inhibited by sequence-specific binding of the inhibitory nucleic acid to its target. Certain inhibitory RNAs, such as short hairpin RNA (shRNA) and short interfering RNA (siRNA), utilize sequence complementarity to target an mRNA for destruction. When appropriately targeted via its nucleotide sequence to a specific mRNA in cells, the inhibitory RNA specifically suppresses target gene expression, reducing the cellular level of the corresponding target mRNA and decreasing the level of protein encoded by such mRNA.

Inhibitory nucleic acids can be single-stranded or double-stranded. Examples of inhibitory nucleic acids include antisense DNA and RNA oligonucleotides, siRNA, shRNA, and micro-RNA. As used herein, the term “knock-down” or “knock-down technology” refers to a technique of gene silencing in which the expression of a target gene or gene of interest is reduced as compared to the gene expression prior to the introduction of an inhibitory RNA, such as an shRNA, which can lead to the inhibition of production of the target gene product. For example, the expression may be reduced by 0.1, 0.5, 1, 5, 10, 15, 20, 25, 30, 35, 40, 45, 50, 55, 60, 65, 70, 75, 80, 85, 90, 95, or even 99%. The expression may be reduced by any amount (%) within those intervals, such as for example, 2-4, 11-14, 16-19, 21-24, 26-29, 31-34, 36-39, 41-44, 46-49, 51-54, 56-59, 61-64, 66-69, 71-74, 76-79, 81-84, 86-89, 91-94, 96, 97, 98 or 99. Reduction of gene expression can be statistically significant, as measured, for example, by a student's T test or other known statistical method, compared to unaltered or wild-type gene expression. Knock-down of gene expression can be directed by techniques known in the art, such as by the use of inhibitory RNA or by the use of genomic editing, such as by CRISPR or TALENs.

When an antisense nucleic acid sequence has sufficient complementarity to an mRNA target sequence, the antisense sequence will specifically bind to the target portion of an mRNA encoding polypeptide, thus inhibiting translation of the target mRNA. The inhibitory antisense sequence typically will have no more than 1, 2, 3, 4, 5, 6, 7, 8, 9, or 10 base mismatches with the target sequence. In many instances, it may be desirable for the sequences of the nucleic acids to be exact matches, i.e. be completely complementary to the sequence to which the oligonucleotide specifically binds, and therefore have zero mismatches along the complementary stretch. Highly complementary sequences will typically bind quite specifically to the target sequence region of the mRNA and can therefore be highly efficient in inhibiting the translation of the target mRNA sequence into polypeptide product. See, for example, U.S. Pat. No. 7,416,849.

In particular embodiments, substantially complementary oligonucleotide sequences are greater than about 80 percent complementary (or ‘% exact-match’) to the corresponding mRNA target sequence to which the oligonucleotide specifically binds, and will, more preferably be greater than about 85 percent complementary to the corresponding mRNA target sequence to which the oligonucleotide specifically binds. In certain aspects, it will be desirable to have even more substantially complementary oligonucleotide sequences for use in the practice of the disclosure, and in such instances, the oligonucleotide sequences are greater than about 90 percent complementary to the corresponding mRNA target sequence to which the oligonucleotide specifically binds, and may in certain embodiments be greater than about 95 percent complementary to the corresponding mRNA target sequence to which the oligonucleotide specifically binds, and even up to and including 96%, 97%, 98%, 99%, and even 100% exact match complementary to the target mRNA to which the designed oligonucleotide specifically binds. See, for example, U.S. Pat. No. 7,416,849. Percent similarity or percent complementary of any nucleic acid sequence may be determined, for example, by utilizing computer programs known in the art.

As used herein, the terms “small interfering” or “short interfering RNA” or “siRNA” refer to an RNA duplex of nucleotides that is targeted to a desired gene and is capable of inhibiting the expression of a gene with which it shares homology. The RNA duplex comprises two complementary single-stranded RNAs of 15, 16, 17, 18, 19, 20, 21, 22, 23, 24, or 25 nucleotides that form 15, 16, 17, 18, 19, 20, 21, 22, 23, 24, or 25 base pairs and possess 3′ overhangs of two nucleotides. The RNA duplex is formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the siRNA duplex is less than 30 nucleotides. The duplex can be 29, 28, 27, 26, 25, 24, 23, 22, 21, 20, 19, 18, 17, 16, 15, 14, 13, 12, 11 or 10 nucleotides in length. The length of the duplex can be 17-25 nucleotides in length. The duplex RNA can be expressed in a cell from a single construct.

As used herein, the term “shRNA” (small hairpin RNA) refers to an RNA duplex wherein a portion of the siRNA is part of a hairpin structure (shRNA). In addition to the duplex portion, the hairpin structure may contain a loop portion positioned between the two sequences that form the duplex. The loop can vary in length. In some embodiments the loop is 6, 7, 8, 9, 10, 11, 12 or 13 nucleotides in length. The hairpin structure can also contain 3′ or overhang portions. In some aspects, the overhang is a 3′ or a 5′ overhang 0, 1, 2, 3, 4 or 5 nucleotides in length. In one aspect of this disclosure, a nucleic acid construct encodes a small hairpin RNA, comprising a sense region, a loop region and an antisense region. Following expression, the sense and antisense regions form a duplex. It is this duplex, forming the shRNA, which hybridizes to, for example, the Salvador (SAV1) mRNA and reduces expression of SAV1.

A “nucleic acid construct” is a synthesized nucleic acid molecule comprising one or more functional nucleotide sequences. Nucleic acid constructs can comprise, for example, nucleic acid sequences required to express a gene product in a cell, including coding sequences and/or regulatory sequences. A “coding sequence” is a nucleotide sequence that encodes a protein or RNA. A coding sequence can also be referred to a cistron. Accordingly, a multi-cistronic nucleic acid construct comprises more than one coding sequence. A “regulatory sequence” is a nucleotide sequence that can increase or decrease expression of a coding sequence. Examples of regulatory sequences include promoters, enhancers, silencers, operators, and untranslated regions (UTRs)

As used herein, the term “operably linked” refers to the association of nucleic acid sequences on a polynucleotide so that the function of one of the sequences is affected by another. For example, a regulatory DNA sequence is said to be “operably linked to” a DNA sequence that codes for an RNA (“an RNA coding sequence” or “shRNA encoding sequence”) or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. An RNA coding sequence refers to a nucleic acid that can serve as a template for synthesis of an RNA molecule such as an siRNA and an shRNA. Preferably, the RNA coding region is a DNA sequence.

“Regulatory elements” are nucleic acid sequences involved in regulating gene expression, and include promoters, enhancers, silencers, and response elements. As used herein, the term “promoter” refers to a nucleotide sequence, usually upstream (5′) to its coding sequence, which directs and/or controls the expression of the coding sequence by providing the recognition for RNA polymerase and other factors required for proper transcription. “Promoter” can include a minimal promoter that is a short DNA sequence comprised of a TATA-box and other sequences that serve to specify the site of transcription initiation, to which regulatory elements are added for control of expression. “Promoter” can also refer to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that stimulates promoter activity and may be an innate element of the promoter or a heterologous element inserted to enhance the level or tissue specificity of a promoter. It is capable of operating in both orientations (sense or antisense), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

As used herein, the term “reporter element” or “marker” is meant a polynucleotide that encodes a polypeptide capable of being detected in a screening assay. Examples of polypeptides encoded by reporter elements include, but are not limited to, lacZ, GFP, luciferase, and chloramphenicol acetyltransferase. See, for example, U.S. Pat. No. 7,416,849. Many reporter elements and marker genes are known in the art and envisioned for use in the compositions and methods of the disclosure.

The terms “subject” and “individual” are used interchangeably and typically comprise a mammal, in certain embodiments a human or a non-human primate. While the compositions and methods are described herein with respect to use in humans, they are also suitable for animal, e.g., veterinary use. Thus certain illustrative organisms include, but are not limited to humans, non-human primates, canines, equines, felines, porcines, ungulates, lagomorphs, and the like. Accordingly, certain embodiments contemplate the compositions and methods described herein for use with domesticated mammals (e.g, canine, feline, equine), laboratory mammals (e.g, mouse, rat, rabbit, hamster, guinea pig), and agricultural mammals (e.g, equine, bovine, porcine, ovine), and the like.

As used herein, the phrase “subject in need thereof” or “individual in need thereof” refers to a subject or individual that suffers or is at a risk of suffering (e.g, pre-disposed such as genetically pre-disposed, or subjected to environmental conditions that pre-dispose, etc.) from a symptom, disease, or condition.

An “effective amount” of an active agent, such as an inhibitory RNA, is an amount sufficient to carry out a specifically stated purpose, such as to inhibit SAV1 expression.

As used herein, the term “pharmaceutically composition” means that the compound is physiologically acceptable and not unacceptably toxic, with no inhibitory effects on the action of an active ingredient when administered to a subject. Such composition can be sterile and can comprise a pharmaceutically acceptable carrier. Suitable pharmaceutical compositions can comprise one or more of a buffer, a surfactant, a stabilizing agent, a preservative, and/or other solubilizing or dispersing agents.

Terms such as “treating” or “treatment” or “to treat” or “alleviating” or “to alleviate” refer to measures that cure, slow down, lessen symptoms of, and/or halt progression of a diagnosed pathologic condition or disorder. Thus, those in need of treatment include those already with the disorder. In certain embodiments, a subject is successfully “treated” for a disease or disorder according to the methods provided herein if the subject shows, e.g., total, partial, or transient alleviation or elimination of symptoms associated with the disease or disorder.

The terms “reduce,” “inhibit,” “diminish,” “suppress,” and grammatical equivalents (including “lower,” “smaller,” etc.) refer to a measurable decrease, in some cases, a statistically significant decrease, in occurrence or activity, including full blocking of the occurrence or activity. For example, “inhibition” can refer to a decrease of about 10%, 20%, 30%, 40%, 50%, 60%, 70%, 80%, 90% or 100% in activity or occurrence. These terms can be used relative to a “control” that has not been subjected to a particular treatment, e.g., a method of the disclosure. In one example, the control can be an untreated sample or subject. In another example, the control can be a sample or subject that has received a different treatment from the treated sample or subject. In some embodiments, a “treated” sample or subject is one that has been subjected to a method of the disclosure.

As used herein, the term “vector” refers to a viral or non-viral nucleic acid sequence that is capable of replication in a host cell (with or without helper sequences, such as packaging sequences), including a plasmid, cosmid, phage, bacteria, yeast, or binary vector. The vector can be double- or single-stranded, linear or circular, and optionally, self-transmissible or mobilizable. A vector can transform prokaryotic or eukaryotic host cells either by integration into the cellular genome or extrachromosomally (e.g., autonomous replicating plasmid with an origin of replication). Viral vectors prepared, for example, from retroviruses, including lentiviruses, adenoviruses, adeno-associated viruses, and envelope-pseudotyped viruses. Examples of lentiviral vectors include equine infectious anemia virus (EIAV), human immunodeficiency virus (HIV), simian immunodeficiency virus (SIV), visna/maedi virus (VMV), caprine arthritis-encephalitis virus (CAEV), equine infectious anaemia virus (EIAV), feline immunodeficiency virus (FIV), and bovine immunodeficiency virus (BIV). Vectors can be complexed with lipids, such as encapsulated in liposomes, or associated with cationic condensing agents.

### II. General Embodiments

The inventors developed a novel approach of myofiber-guided recreation of a regenerative microenvironment that potentially has translational implications. To create a regenerative milieu for inducing a coordinated angiogenic and myogenic program in vivo, the inventors modulated growth-suppressing Hippo signaling events in the myofibers, which are the key cellular constituents of the muscle stem cell niche that harbor SCs. The inventors investigated whether downregulation of the myogenic Hippo pathway can provide signals for vasculature remodeling and SC activation. The inventors found that SAV1 KD in myofibers of mouse ischemic muscle promoted endothelial cell proliferation, accelerated perfusion recovery, and improved mouse treadmill exercise endurance. After cardiotoxin-induced acute injury, the regenerative area of TA muscle had a larger myofiber cross-sectional area and more capillaries in AAV9 SAV1 KD mice than in control mice, reflecting a better regenerative capability of the muscle after SAV1 KD treatment. Supporting the inventors' in vivo findings, SCs grown in the SAV1 KD-myotube conditioned medium showed a higher percentage of EdU incorporation than those grown in control-myotube conditioned medium, indicating the paracrine influence on cell proliferation.

Accumulating clinical data indicate that effective treatment of ischemic limbs requires not only the availability of angiogenic factors to stimulate EC angiogenesis, but also a myogenic switch to activate the skeletal muscle SCs for promoting concurrent myogenesis. The unique SC niche comprises the spatial relationship between the myofibers and the associated SCs and ECs. The myofibers provide the signals required to establish a functional niche for activating SCs and stimulating growth of the surrounding vasculature. Thus, modulating the intrinsic properties of the myofibers will provide signals to recreate a supportive niche for promoting skeletal muscle regeneration. Indeed, when the inventors modulated Hippo signaling pathway in myofibers, they found paracrine interactions between myofibers and their associated SCs. The extracellular factors secreted from myofibers facilitate SC activation and promoted myogenesis.

Gene therapy for leg ischemia has been focused on angiogenic therapeutics. In this approach, plasmids or adenovirus vectors are used to release genes encoding various growth-factors into ischemic legs to improve blood flow. Such studies have included using genes encoding vascular endothelial growth factor (VEGF), fibroblast growth factor (FGF), and hypoxia-inducible factor 1-alpha (HIF-1α).^{2,40} Meta-analysis of these randomized control trials has shown no clear difference in major amputation and amputation-free survival rates between gene therapy and placebo treatment. 2 The major limitations of this approach include the gene delivery system (carriers), transfection efficiency, and the inefficiency of targeting a single growth factor to compensate for tissue ischemia. Plasmid vectors are not efficient in delivering exogenous genes into cells. They are non-replication episomes and cannot achieve sufficient level and long-term duration of therapeutic transgene expression.⁴¹ Although growth factors such as VEGF and FGF enhance angiogenesis, it is unlikely that a single angiogenic factor can control the complicated biological process of new blood vessel formation in ischemic tissue.^{42,43} Targeting a regulatory gene/pathway that coordinates the cascade of events involved in vascularization may be more practical than targeting a single angiogenic growth factor. Moreover, several issues must be addressed before efficient and robust gene therapeutic modalities can be implemented to achieve better clinical outcomes for treating leg ischemia. In particular, a suitable carrier must be used to deliver genetic material to the area of interest, and the type of cells or tissues to be targeted must be identified.

Recombinant AAV vectors infect both dividing and nondividing cells and persist without directly integrating into the host genome. Because these vectors do not trigger an immune response, they are considered nonpathogenic and safe and have emerged as efficacious, long-term, and tissue-specific gene delivery systems in the clinical setting.^{44,45} In the current study, the inventors integrated AAV9 and small-interfering RNA (siRNA) therapeutics to target SAV1, the Hippo pathway adapter. The inventors used a novel AAV9 vector with a minimally sized skeletal muscle-specific expression cassette to accommodate its packaging and to induce pooled miR30-based shRNAs for SAV1 knockdown in mouse myofibers. The inventors observed myogenesis, perfusion recovery, and improved skeletal muscle strength in mouse ischemic hindlimbs. This approach overcomes the limitations of low transfection efficiency in siRNA therapeutics and tissue specificity.

The inventors used an AAV9 vector delivery system that induced a “Hippo downregulation” condition in the myofibers to create a regenerative microenvironment, which ensures SC activation, myogenesis, EC angiogenesis, and neovascularization. This strategy is illustrated in FIG. 1. In particular, the Hippo pathway adaptor, Salvador, was knocked down by using an adeno-associated virus 9 (AAV9) vector expressing a miR30-based triple short-hairpin RNA (shRNA), controlled by a muscle-specific promoter. The inventors validated the feasibility of this novel approach in both mouse hindlimb ischemia and skeletal muscle regeneration models. In a mouse hindlimb-ischemia model, AAV9 Salvador shRNA administration in ischemic muscles induced nuclear localization of the Hippo effector YAP, accelerated perfusion restoration, and increased exercise endurance. Intravascular lectin labeling of the vasculature confirmed enhanced angiogenesis. Using 5-ethynyl-2′-deoxyuridine to label replicating cellular DNA in vivo, it was found that Salvador knockdown concurrently increased paired box transcription factor Pax7+muscle satellite cell and CD31+ endothelial cell proliferation in ischemic muscles.

In addition to improving angiogenesis, it is shown herein that Hippo pathway inhibition after acute muscle injury or damage improved muscle function in a mouse model of lower extremity ischemia. Two weeks after delivery of AAV9 Salvador shRNA into injured muscles, the distribution of regenerative myofibers shifted toward a larger cross-sectional area compared with mice receiving AAV9 control shRNA. In an aged mouse (26-month-old) cohort, AAV9 Salvador shRNA treatment increased the number of regenerative myofibers containing a larger cross-sectional area as compared to control treatment.

Taken together, the current disclosure provides that Hippo inhibition via SAV1 knockdown in myogenic cells promotes cell proliferation, perfusion restoration, and skeletal muscle regeneration in models of mouse hindlimb ischemia and skeletal muscle injury. Accordingly, the disclosure provides methods that could overcome the primary hurdle in the age-associated decline in skeletal muscle regeneration and the current limitations of vascular treatment in ischemic muscle regeneration.

One embodiment demonstrated herein is a unique, but exemplary, set of three shRNAs that specifically target the Hippo pathway member SAV1. The shRNAs provide selective reduction in SAV1 mRNA levels similar to a genetic knockout in a mouse model. In specific embodiments, the shRNAs can be delivered using an AAV9 (Adeno Associated Virus serotype 9) vector. Particular embodiments of the disclosure contemplate the shRNA sequence of nucleotides specific to target SAV1.

### III. Salvador

The inventors inhibited the Hippo pathway by downregulating the adaptor protein, Salvador (SAV1), because this pathway provides growth restriction signals that inhibit tissue regeneration. 31 The role of the Salvador-Warts-Hippo (SWH) pathway in controlling cell growth has been studied extensively in Drosophila.³² The activation of the SWH pathway results in smaller imaginal disc size with reduced cell proliferation. SAV serves as a scaffold protein that binds to both Warts and Hippo kinases to facilitate downstream phosphorylation events by which the SWH pathway activity is upregulated.³² As a positive component for activating the SWH pathway, SAV is also an evolutionarily conserved member of the SWH signaling family. In mammalian cells, SAV1 physically interacts with MST1/2 (Hippo in Drosophila) kinases to activate LATS 1/2 (Warts in Drosophila) via phosphorylation. The active LATS 1/2 phosphorylates the downstream effector YAP, leading to its nucleus exclusion. Thus, Hippo pathway activation inhibits YAP as a transcriptional co-activator to promote growth-related gene expression.^{31,33} Research has shown that SAV1 is required for Hippo kinase cascade-mediated control of organ size.³⁴ Liver-specific knockout of SAV1 in mice significantly increased liver size and the proliferation index. The enhanced cell proliferation was confirmed by increased nuclear incorporation of BrdU in vivo.³⁴ In a mouse conditional knockout model, heart-specific SAV1 knockout resulted in an enlarged heart and reduced YAP phosphorylation.³¹ These findings highlight the critical role of SAV1 in regulating Hippo pathway activity.

The gene may be referred to as salvador homolog 1, Salv, SAV1, SAV, WW45, or WWP4. A representative nucleic acid is provided at GenBank® Accession No. CR457297.1, and a representative protein sequence is provided at GenBank® Accession No. Q9H4B6. The cDNA sequence of human Salvador is set forth in SEQ ID NO: 1 (FIG. 2).

The gene encodes a protein which includes 2 WW domains (a modular protein domain containing two conserved tryptophan residues, which mediates specific interactions with protein ligands) and a coiled-coil region. It is ubiquitously expressed in adult tissues. It also includes a SARAH (Sav/Rassf/Hpo) domain at the C terminus (three classes of eukaryotic tumor suppressors that give the domain its name). In the Say (Salvador) and Hpo (Hippo) families, the SARAH domain mediates signal transduction from Hpo via the Say scaffolding protein to the downstream component Wts (Warts); the phosphorylation of Wts by Hpo triggers cell cycle arrest and apoptosis by down-regulating cyclin E, Diap 1 and other targets. The SARAH domain may also be involved in dimerization.

### IV. Inhibitory Nucleic Acids that Target SAV1

The methods of the disclosure utilize one or more inhibitory nucleic acids target SAV1, such that expression of SAV1 is detectably reduced. The inhibitory nucleic acid may be DNA or RNA. In specific embodiments the nucleic acid is an inhibitory RNA, such as shRNA. In some embodiments, the inhibitory nucleic acid targets a sequence that encodes the N-terminal region of the Sav1 protein, sequence that encodes the middle of the SAV1 protein, or sequence that encodes the C-terminal region of the SAV1 protein. In one embodiment, the inhibitory RNA has or is encoded by the nucleotide sequence aagtacgtgaagaaggagacg (SEQ ID NO: 2). In one embodiment, the inhibitory RNA has or is encoded by the nucleotide sequence aagatttaccccttcctcctg (SEQ ID NO: 3). In one embodiment, the inhibitory RNA has or is encoded by the nucleotide sequence aattcctgactggcttcaggt (SEQ ID NO: 4).

In one embodiment, the inhibitory RNA is an shRNA having a “hairpin” or stem-loop RNA molecule, comprising a sense region, a loop region, and an antisense region complementary to the sense region. In other embodiments the inhibitory RNA is an siRNA comprising two distinct, complementary RNA molecules (strands) that are non-covalently associated via base pairing to form a duplex. See, for example, U.S. Pat. No. 7,195,916.

In particular cases, the inhibitory RNA is an shRNA. The shRNA is a single-stranded RNA molecule that forms a stem-loop structure in vivo, and it may be from about 40, 41, 42, 43, 44, 45, 46, 47, 48, 49, 50, 51, 52, 53, 54, 55, 56, 57, 58, 59, or 60 nucleotides (nt) in length to 120, 121, 122, 123, 124 125, 126, 127, 128, 129, 130, 131, 132, 133, 134, 135, 136, 137, 138, 139 or 140 (nt) in length. The duplex portion of the stem-loop structure can be less than 30 nucleotides in length, such as 10, 11, 12, 13, 14, 15, 16, 17, 18, 19, 20, 21, 22, 23, 24, 26, 27, 28, or 29 nucleotides in length, including ranges within these lengths. The complementary RNA sequences that create the double-stranded stem by base pairing are preferably 19- to 29-nt-long. The shRNA can further comprise an overhang region. Such an overhang may be a 3′ overhang region or a 5′ overhang region. The overhang region may be, for example, 1, 2, 3, 4, 5, or 6 nucleotides in length. A loop structure containing, for example, from 4-10 nucleotides (i.e., 4, 5, 6, 7, 8, 9, 10) connects the two complementary RNA sequences that form the stem. Transcription and synthesis of shRNA in vivo is directed by Pol III promoter, and then the resulting shRNA is cleaved by Dicer, an RNase III enzyme, to generate mature siRNA. The mature siRNA enters the RISC complex. Thus, in specific embodiments, shRNA for inhibition of SAV1 expression in accordance with the present disclosure contains both sense and antisense nucleotide sequences.

In certain embodiments, the nucleic acid comprises the sequence of SEQ ID NO:2 (or SEQ ID NO:3 or 4) and further comprises an antisense sequence of SEQ ID NO:2 (or, respectively, SEQ ID NO:3 or 4), wherein when the sequence and the antisense sequence are hybridized together to form a duplex structure, the sequence and the antisense sequence are separated by a loop structure.

In a one embodiment, the nucleic acid construct comprises a polynucleotide sequence encoding an shRNA operably linked to a promoter. In one embodiment, the shRNA comprises a first segment, a second segment located immediately 3′ of the first segment, and a third segment located immediately 3′ of the second segment, wherein the first and third segments can each be less than 30 base pairs in length and can each be more than 10 base pairs in length. The first segment and the third segment are complementary to one another, one comprising an antisense sequence and the other comprising a sense sequence, relative to a target sequence. The second segment, located immediately 3′ of the first segment, encodes a loop structure.

The inhibitory RNA molecules for use in the disclosure may be substantially identical (for example, at least about 80%, 81%, 82%, 83%, 84%, 85%, 86%, 87%, 88%, 89%, 90%, 91%, 92%, 93%, 94%, 95%, 96%, 97%, 98%, or 99% identical) to a SAV1 sequence and/or to any one of SEQ ID NO: 2, SEQ ID NO: 3, or SEQ ID NO: 4. In specific embodiments, there are no more than 5 mismatches between the sequence of the inhibitory RNA and the target SAV1 sequence. In specific embodiments, a minimum of 18 bp homology is utilized for the region of complementarity between the inhibitory RNA sequence and its target. Suitable mismatches can be predicted by known algorithms and/or identified by known assays.

In some embodiments, the inhibitory nucleic acid sequence, such as a sequence encoding an inhibitory RNA is comprised in a nucleic acid construct. In particular embodiments, the nucleic acid construct is comprised in a vector, including a viral or non-viral vector. In specific embodiments, the vector is non-integrating, although in other embodiments it is integrating. If mechanisms are included to direct the integration of the vector or a vector segment into the host-cell genome, or to ensure the stability of the transcription vector, the inhibitory nucleic acid can be made stable and heritable.

Viral vectors may be lentiviral, adenoviral, adeno-associated viral, and retroviral, for example. Non-viral vectors include plasmids. In specific embodiments, the inhibitory nucleic acid is comprised in an AAV vector. The AAV vector can be of any serotype, including, for example, AAV2, AAV6, AAV7, AAV8, and AAV9. (Piras et al., 2013) In one embodiment, an AAV9 vector is employed.

For expression of an inhibitory RNA, a promoter is operably linked to the sequence encoding the inhibitory RNA. The nucleic acid construct of the present disclosure may further comprise various expression regulatory sequences such as an optional operator sequence for controlling transcription, and sequences controlling the termination of transcription.

The promoter used in the present disclosure can be a constitutive promoter that constitutively induces the expression of a target gene, or an inducible promoter that induces the expression of a target gene at a given position and time point. Specific examples include U6 promoter, cytomegalovirus (CMA) promoter, respiratory syncytial virus (RSV) promoter, SV40 promoter, CAG promoter (Hitoshi Niwa et al., Gene, 108:193-199, 1991; and Monahan et al., Gene Therapy, 7:24-30, 2000), CaMV 35S promoter (Odell et al., Nature 313:810-812, 1985), Rsyn7 promoter (U.S. patent application Ser. No. 08/991,601), ubiquitin promoter (Christensen et al., Plant Mol. Biol. 12:619-632, 1989), ALS promoter (U.S. patent application Ser. No. 08/409,297), and the like. Examples of promoters are disclosed in U.S. Pat. Nos. 5,608,149, 5,608,144, 5,604,121, 5,569,597, 5,466,785, 5,399,680, 5,268,463, 5,608,142, etc.

In certain instances, the promoter can be a tissue-specific or cell-specific promoter, such as a myofiber-specific or skeletal muscle-specific promoter. Promoters suitable for use in skeletal muscle include, for example, promoters from muscle creatine kinase (MCK), desmin, actin, troponin (such as troponin T/I or chicken cardiac troponin T (cTnT)), myosin heavy chain, myosin light chain, myoglobin, NCX1, and hybrids thereof. Specific example of promoters include rat ventricle-specific cardiac myosin light chain 2 (MLC-2v) promoter, cardiac muscle-specific alpha myosin heavy chain (MHC) gene promoter, and cardiac cell-specific minimum promoter from −137 to +85 of NCX1. In one embodiment, the promoter is a cardiac troponin T promoter.

Where a nucleic acid construct comprises a polycistronic nucleic acid, i.e., a nucleic acid encoding more than one inhibitory RNA, the inhibitory RNAs can be under the control of single promoter or multiple promoters. In one embodiment, each inhibitory RNA is regulated by a separate promoter. In some instances, the nucleic acid construct comprises short inverted repeats separated by a small number of (e.g., 3, 4, 5, 6, 7, 8, 9) nucleotides that direct the transcription of inhibitory RNAs.

Accordingly, the amount of inhibitory RNA generated in a target cell can be regulated by controlling such factors as the nature of the promoter used to direct transcription of the nucleic acid sequence, (i.e., whether the promoter is constitutive or regulatable, strong or weak) and the number of copies of the nucleic acid sequence encoding the inhibitory RNA that are in the cell.

A nucleic acid construct for use in the disclosure can comprise one or more regulatory elements. In specific cases, each inhibitory RNA sequence can be regulated by the same regulatory sequence or each inhibitory RNA sequence can be regulated by a different regulatory sequence. In some embodiments, the nucleic acid construct encoding an inhibitory RNA for use in the disclosure comprises a post-transcriptional regulatory element (PRE). Examples of PREs include the woodchuck hepatitis virus PRE (WPRE), hepatitis B virus PRE, and Intron A of human cytomegalovirus immediate early gene. See Sun et al. 2009 and Mariati et al. 2010 for further examples and details. In a particular embodiment, the PRE is a WPRE.

In certain embodiments, the nucleic acid construct comprises one or more additional features, such as a 5′ untranslated region (UTR), a 3 UTR, inverted terminal repeats (ITRs), and/or a polyadenylation signal, such as a synthetic minimal polyadenylation signal. (See van der Velden et al. 2001.)

The nucleic acid construct can comprise microRNA (miRNA) sequences, for example, miRNA-30 sequences. This enables the triple shRNAs to be processed in the target cell as an miRNA, rather than as a shRNA, by an RNA polymerase type II promoter, enhancing expression efficiency of the shRNAs. (See Dow et al. 2012.)

In one aspect, methods of the disclosure utilize multiple nucleic acid constructs, each encoding a different inhibitory RNA, such as an shRNA, targeted to a different region of the SAV1 nucleic acid sequence. A single nucleic acid construct can encode multiple inhibitory RNAs targeted to different areas of the same gene; for example, comprising two or more SEQ ID NO: 2, SEQ ID NO: 3, or SEQ ID NO: 4. In another aspect, a single nucleic acid can encode multiple copies of the same inhibitory RNA. In a further aspect, a single nucleic acid construct can encode multiple copies of multiple inhibitory RNAs, for example, multiple copies of SEQ ID NO: 2, multiple copies of SEQ ID NO: 3, and/or multiple copies of SEQ ID NO: 4 in any combination. Each nucleic acid construct can be comprised in a different vector.

The nucleic acid construct can further comprise one or more marker genes, such as a selectable marker, and/or one or more reporter genes. The marker genes or reporter genes provide a method to track expression of one or more linked genes. The marker genes or reporter genes, upon expression within the cell, provide products, usually proteins, detectable by spectroscopic, photochemical, biochemical, immunochemical, chemical, or other physical means. Gene expression products, whether from the gene of interest, marker genes or reporter genes may also be detected by labeling. Labels envisioned for use in the compositions and methods of the disclosure include, but are not limited to, fluorescent dyes, electron-dense reagents, enzymes (for example, as commonly used in an ELISA), biotin, digoxigenin, or haptens and proteins which can be made detectable, e.g., by incorporating a radiolabel into the peptide or used to detect antibodies specifically reactive with the peptide. See, for example, U.S. Pat. No. 7,419,779.

In a particular embodiment, methods of the disclosure utilize a nucleic acid construct comprising: (i) an shRNA comprising an inhibitory nucleic acid having the nucleotide sequence set forth in SEQ ID NO: 2, (ii) an shRNA comprising an inhibitory nucleic acid having the nucleotide sequence set forth in SEQ ID NO: 3, and (iii) an inhibitory nucleic acid having the nucleotide sequence set forth in SEQ ID NO: 4; wherein the shRNAs are operably linked to at least one promoter. The promoter is preferably a muscle-specific promoter, such as a cardiac troponin T promoter. The nucleic acid construct can be comprised in a vector, for example, a viral vector. Specific embodiments include an AAV vector, such as an AAV9 vector.

Standard recombinant DNA and molecular cloning techniques used in the present disclosure are well known in the art and can be found in the following literature: Sambrook, J., Fritsch, E. F. and Maniatis, T., Molecular Cloning: A Laboratory Manual, 2nd ed., Cold Spring Harbor Laboratory: Cold Spring Harbor, N.Y. (1989); Silhavy, T. J., Bennan, M. L. and Enquist, L. W., Experiments with Gene Fusions, Cold Spring Harbor Laboratory: Cold Spring Harbor, N.Y. (1984); and Ausubel, F. M. et al., Current Protocols in Molecular Biology, published by Greene Publishing Assoc. and Wiley-Interscience (1987).

### V. Inhibitory Nucleic Acid Synthesis

Inhibitory nucleic acids can be prepared by methods known in the art for the synthesis of DNA and RNA molecules. These include techniques for chemically synthesizing oligodeoxy-ribonucleotides and oligo-ribonucleotides well known in the art such as, for example, solid phase phosphoramidite chemical synthesis. Alternatively, RNA molecules can be generated by in vitro and in vivo transcription of DNA sequences encoding the inhibitory RNA molecule. Such DNA sequences may be incorporated into a wide variety of vectors that incorporate suitable RNA polymerase promoters such as the T7 or SP6 polymerase promoters. Alternatively, antisense cDNA constructs that synthesize inhibitory RNA constitutively or inducibly, depending on the promoter used, can be introduced stably into cell lines.

Inhibitory RNA molecules can be chemically synthesized using appropriately protected ribonucleoside phosphoramidites and a conventional DNA/RNA synthesizer. Custom synthesis services are available from commercial vendors such as Ambion (Austin, Tex., USA) and Dharmacon Research (Lafayette, Colo., USA). See, for example, U.S. Pat. No. 7,410,944.

Various well-known modifications to the DNA molecules can be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone. An antisense nucleic acid of the disclosure can be constructed using chemical synthesis or enzymatic ligation reactions using procedures known in the art. An antisense oligonucleotide can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

The inhibitory RNA molecules for use in methods of the disclosure can be various modified equivalents of the SAV1 inhibitory RNAs disclosed herein. A “modified equivalent” means a modified form of a particular inhibitory RNA molecule having the same target-specificity (i.e., recognizing the same mRNA molecules that complement the unmodified particular inhibitory RNA molecule). Thus, a modified equivalent of an unmodified inhibitory RNA molecule can have modified ribonucleotides, that is, ribonucleotides that contain a modification in the chemical structure of an unmodified nucleotide base, sugar and/or phosphate (or phosphodiester linkage). See, for example, U.S. Pat. No. 7,410,944.

### VI. Methods of Use

Some embodiments of the disclosure concern methods and compositions for regenerating skeletal muscle. Regeneration can include the regeneration of myofibers, proliferation and differentiation (myogenesis) of satellite cells and angiogenesis in adjacent endothelial cells, along with enhanced neovascularization and increased blood flow. The skeletal muscle may be in need of regeneration due to disease, underlying genetic condition, age, and/or trauma, for example. In specific embodiments, the skeletal muscle has damage, atrophy, apoptosis, necrosis, and/or autophagy, such as with ischemia of the lower extremities, for example.

Ischemia is a reduction or restriction of the blood supply to cells or tissues, resulting in hypoxia. Such lack of blood flow to the lower extremities, for example, in peripheral vascular disease, such as peripheral arterial disease, causes oxygen and nutrient deprivation in ischemic skeletal muscles, leading to functional impairment. Treatment options for muscle regeneration in this scenario are lacking. The inventors demonstrate that selectively targeting the Hippo pathway in myofibers, which provide architectural support for muscle stem cell niches, facilitates functional muscle recovery in ischemic extremities by promoting angiogenesis, neovascularization, and myogenesis. Specifically, inhibiting the Hippo pathway in myogenic cells alters the composition of the myofibers' secretome and enriches the local milieu with factors that stimulate and promote the proliferation and differentiation (myogenesis) of satellite cells (SCs) and angiogenesis in adjacent endothelial cells, along with enhancing neovascularization.

Accordingly, in one embodiment, the disclosure provides a method for treating limb ischemia, especially lower limb ischemia in a subject. Treatment of limb ischemia, for example, chronic or acute limb ischemia can include, but is not limited to, amelioration of pain, pallor (paleness of the skin), paresthesias (abnormal sensations), or cold feeling in the limb; improved distal pulse in the limb; decreased paralysis of the limb; or improved blood flow in the limb. Methods of assessing limb ischemia and its symptoms include pulse examination, measurement of local temperature, evaluation of skin color, Doppler evaluation, ultrasound, and angiography.

In a particular embodiment, the methods of the disclosure promote myofiber regeneration in aged subjects. An “aged” subject is an individual who is at least 50, 55, 60, 65, or 75 years of age. Aged subjects in need of myofiber regeneration include those with sarcopenia, skeletal muscle damage, atrophy, apoptosis, necrosis, and/or autophagy.

Methods of the disclosure comprise delivering to skeletal muscle cells or tissue an inhibitory nucleic acid that targets SAV1. Targeted inhibition of SAV1 by the methods of the disclosure can cause several physiological effects. For example, skeletal myofibers are able to activate signals for SC proliferation and self-renewal. In addition, nuclear localization of the Hippo effector YAP is induced in existing skeletal myofibers, perfusion restoration is accelerated, and/or exercise endurance increased in an individual to whom the inhibitory nucleic acid is administered. Proliferation of paired box transcription factor Pax7+ muscle satellite cells and CD31+ endothelial cells can be increased in ischemic muscles. The distribution of regenerative myofibers shifts toward a larger cross-sectional area, and the number of regenerative myofibers containing a larger cross-sectional area is increased in damaged muscles.

Methods of the disclosure can result in increased mass of an affected muscle, compared with the state of the affected muscle prior to administration of at least one inhibitory nucleic acid targeting SAV1. Methods of the disclosure can result in collateralization of blood vessels in an affected muscle, compared with the state of the affected muscle prior to administration of at least one inhibitory nucleic acid targeting SAV1.

Methods of assessing myogenesis, skeletal muscle regeneration and/or muscle mass include, for example, computerized tomography, magnetic resonance imaging (MRI), dual-energy X-ray absorptiometry (DXA), bioimpedance analysis, and biopsy and histology. Methods of assessing angiogenesis, neovascularization, and/or blood flow include, for example, pulse examination and, measurement of local temperature, evaluation of skin color, Doppler evaluation, ultrasound, angiography, and biopsy and histology.

Inhibitory nucleic acids can be delivered directly to target cells, for example, in the form of single-stranded RNA or double-stranded RNA.^{50,51} Alternatively, inhibitory RNA can be delivered into target cells, using a DNA construct comprised in a vector, from which RNA can be transcribed. In specific embodiments, the inhibitory RNA is expressed in a target skeletal muscle cell, preferably a human skeletal muscle cell. As used herein, “skeletal muscle cells” include myocytes and satellite cells.

Vectors comprising an inhibitory RNA can be delivered to an individual systemically or locally. Systemic administration is preferably via the parenteral route, for example, intraperitoneal, intravenous, or subcutaneous. Local administration is preferably via injection at a desired organ or tissue site, for example, into muscle tissue, such as injured or ischemic muscle tissue.

Inhibitory nucleic acids can be delivered in a pharmaceutical composition. The composition can comprise, in addition to the inhibitory nucleic acid, a pharmaceutically acceptable excipient, carrier, buffer, stabilizer or other materials. Examples of commonly used carriers include normal (isotonic) saline, such as 0.9% saline and dextrose, such as 5% dextrose. The composition can be buffered, for example to a pH of between 4 and 8.

In some embodiments, inhibitory RNA or a nucleic acid construct encoding inhibitory RNA against SAV1 is introduced into skeletal muscle cells or tissues in vitro or ex vivo. Methods for introduction include transfection with calcium chloride or calcium phosphate or polyethylenimine; microprojectile bombardment, electroporation, PEG-mediated fusion, microinjection, liposome-mediated methods, and the like. Optionally, skeletal muscle cells comprising inhibitory RNA can be introduced into a subject in need of skeletal muscle regeneration, for example, as a tissue graft. The grafted tissue can be an allograft, and autograft, or engineered tissue.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Small Hairpin RNA Inhibits SAV1 Expression in Cardiomyocytes

The inventors measured the ability of shRNAs encoded by each of SEQ ID NO: 2, SEQ ID NO: 3, and SEQ ID NO: 4 to suppress endogenous SAV1 expression in neonatal cardiomyocytes. Mouse neonatal primary cardiomyocytes were isolated and cultured in 24-well plates to 60-80% confluence. Cells were transfected with 1 μL siRNA (10 pmol; C F=10 11M) comprising SEQ ID NO: 2, using 3 μL Lipofectamine RNAiMAX reagent (ThermoFisher Scientific). Cells were collected 48 hours after transfection and SAV1 mRNA levels were measured by quantitative RT-PCR. Results are shown in FIG. 3A.

In another experiment, pig SK6 cells were cultured in 6-well plates to 70-90% confluence. Cells were transfected with 1 μg of a lentiviral shRNA plasmid comprising SEQ ID NO: 3 under the control of a U6 promoter (U6-Salv-shmiRNA), using 3 μL Lipofectamine 3000 reagent (ThermoFisher Scientific). Cells were collected 48 hours after transfection and SAV1 mRNA levels were measured by quantitative RT-PCR. Results are shown in FIG. 3B.

In an additional experiment, human AC16 cells were cultured in 6-well plates to 70-90% confluence. Cells were transfected with 1 μg of an AAV shRNA plasmid comprising SEQ ID NO: 4 under the control of a cTnT promoter (AAV-cTnT-Salv-shmiR), using 3 μL Lipofectamine 3000 reagent (ThermoFisher Scientific). Cells were collected 48 hours after transfection and SAV1 mRNA levels were measured by quantitative RT-PCR. Results are shown in FIG. 3C.

All three inhibitory nucleic acid sequences knocked down SAV1 expression by approximately 70%, with SEQ ID NO: 4 displaying the greatest efficiency.

### Example 2

### AAV9 Mediated Myogenic Salvador Knockdown in Ischemic Legs

The inventors first evaluated the expression of the SAV1 and the Hippo signaling downstream effector Yes-associated protein 1 phosphorylation on the serine residue (pYAP, Ser 127) on day 14 after induction of unilateral hindlimb ischemia. When compared to the non-ischemic contralateral gastrocnemius muscle, ischemic muscles showed significantly higher levels of SAV1, total YAP, and pYAP proteins. The ratio of pYAP to YAP did not change (FIG. 4, p<0.05, n=4 mice/group).

Next, the inventors used a muscle-tropic AAV9 vector containing a myogenic-specific cassette under the cardiac troponin T promoter to drive the miR30-based shRNA to knockdown SAV1 in myofibers of the ischemic legs of C57BL/6J mice.¹⁸ Because SAV1 coordinates the Hippo kinase cascade to phosphorylate and inhibit YAP activity, SAV1 knockdown downregulates YAPs^{ser127} phosphorylation, inducing activation and nuclear translocation of YAP.^{18,24} After confirming unilateral hindlimb ischemia at day 3 after double ligation, the inventors injected AAV9 SAV1 or control shRNA (1×1010 viral genomes/mouse) into the gastrocnemius muscle of the ischemic hindlimbs (n=5 mice/group) and collected the muscles 7 days after AAV9 administration. Western blot of muscle lysates and semi-quantitative analysis confirmed SAV1 knockdown and reduced total YAP and pYAP expression (FIG. 5A-5E) after SAV1 shRNA treatment. No significant change was found in the ratio of pYAP to total YAP between control and SAV1 shRNA groups (FIG. 5F). Given that the gastrocnemius muscle comprises a variety of cell types other than muscle fibers, the inventors used immunostaining to detect YAP immunoreactivity within gastrocnemius myofibers. Microscopic immunofluorescence analysis of gastrocnemius muscle sections at 14 days after AAV treatment (n=4 mice/group) revealed more nuclear accumulation of YAP in the myofibers of the AAV9 SAV1 shRNA group than in the AAV9 control shRNA group (FIG. 5G-5I). These results indicate that SAV1 knockdown mediated YAP nuclear localization.

### Example 3

### Salvador Knockdown Accelerates Restoration of Perfusion and Muscular Strength after Hindlimb Ischemia

The inventors examined the effect of SAV1 knockdown on blood flow restoration in ischemic legs. Perfusion was assessed immediately before AAV9 injection into ischemic legs and every 7 days thereafter for up to 9 weeks (FIG. 6A, n=18 mice/group). Before AAV administration, quantification of laser Doppler perfusion images showed a dramatic reduction in blood perfusion in ischemic legs over that seen in their individual contralateral nonischemic legs, and there was no difference in ischemic perfusion reduction between treatment and control groups. These results confirmed the successful induction of ischemia (WO, FIG. 6A, 6B).

At 3 weeks after AAV injection, mice started to respond to SAV1 shRNA therapy. At week 5, perfusion was significantly better in the SAV1 knockdown group than in control group, and this benefit extended to week 9 (FIG. 6A, 6B). On week 9 after AAV9 shRNA delivery, the inventors injected a vascular endothelial marker (tomato lectin, 100 μg/mouse) intravenously into the tail vein 10 minutes before euthanizing the mice (n=4 mice/group). Confocal immunofluorescence imaging showed higher numbers of red fluorescent tomato lectin-labeled vascular structures (i.e., capillaries and arterioles) in the gastrocnemius muscle of the SAV1 knockdown group than in that of the control group (FIG. 6C, 6D).

To assess if improved perfusion associated with AAV9 SAV1 shRNA therapy concurrently increased muscle strength and functional ability, the inventors performed a treadmill exhaustion test in the same mouse cohorts used to measure blood flow. At 7 weeks after AAV9 shRNA injection, mice in the SAV1 knockdown group performed significantly better than control-treated mice (FIG. 6E).

### Example 4

### Salvador Knockdown Promotes Activation of Satellite Cells and Proliferation of Endothelial Cells in Ischemic Hindlimbs

To determine if SAV1 knockdown affected cell proliferation, the inventors pulsed mice with intraperitoneal injections of the nucleotide analog EdU (5-ethynyl-2′-deoxyuridine) to label replicating DNA. The inventors found the number of EdU⁺nuclei increased significantly at 2 weeks after AAV9 SAV1 shRNA injection (FIG. 7A, 7B; 6.61±0.54% vs 2.94±0.33%, SAV1 knockdown vs. control; p<0.05, n=4 mice/group). Then, the inventors evaluated the distribution of EdU+ nuclei around myofibers. In the AAV9 control treatment group, most EdU+ nuclei were found in the interstitial area of skeletal muscle (FIG. 7C, yellow arrows), whereas in the AAV9 SAV1 shRNA treatment group, more EdU+ nuclei were detected at typical SC anatomical locations (FIG. 7C, red arrows). The EdU+ nuclei aligned along the grooves of the periphery of longitudinal skeletal muscle fibers, or Edu+ nuclei were located between the myofiber membrane and the basal lamina of cross-sectional muscle areas. This finding implies ongoing muscle regeneration after SAV1 knockdown in skeletal muscle.

SC activation was further validated by the concurrent nuclear incorporation of EdU and the expression of the transcription factor Pax7. Pax7 expression is specific to SCs and is required for skeletal muscle regeneration.^{25,26} Activated SCs exhibited nuclear colocalization of Pax7 and EdU (FIG. 8A; FIG. 9, yellow arrows), which is contrary to quiescent Pax7+Edu− SCs (FIG. 8A, white arrows). Knockdown of SAV1 significantly increased the percentage of Pax7+EdU+ within EdU+ cell populations per high power field over that seen with control treatment (FIG. 8B; 26.65±3.10% vs 13.25±0.55%, SAV1 KD vs. control; p<0.05, n=4 mice/group). The inventors used CD31 staining to examine ongoing angiogenesis in ischemic limbs. EdU labeling of CD31+ cells indicated endothelial proliferation (FIG. 8C, 8D, purple arrows). In the SAV1 knockdown group, EdU+ nuclei were found along capillary sprouts (FIG. 8C, green arrows). In analyzing collateral vessels, the inventors found EDU+CD31+ endothelial cells in the endothelium, indicating SAV1 knockdown promoted the proliferative activity of collateral vessels (FIG. 8C, orange arrows). This further supports the findings in the inventors' in vivo lectin-vascular labeling and laser Doppler perfusion studies (FIG. 6A-6D).

### Example 5

### Salvador Knockdown Accelerates Myogenesis after Skeletal Muscle Injury

The inventors used a mouse model of cardiotoxin-induced muscle injury to assess whether SAV1 knockdown in myofibers enhanced skeletal muscle regeneration (12 to 15-week-old mice). On day 3 after cardiotoxin injection, AAV was injected into the damaged transverse abdominal (TA) muscle area (FIG. 10A). On day 14 after AAV injection, hematoxylin and eosin staining of TA muscle sections showed that cardiotoxin-induced muscle damage was largely replaced with regenerating myofibers with central nuclei (FIG. 10B). A morphometrical analysis of the cross-sectional areas of regenerating myofibers revealed a difference in the distribution of fiber size between the control and SAV1 knockdown groups (FIG. 10C). The control group had a higher percentage of myofibers with a cross-sectional area <1000 um² than did the SAV1 knockdown group, and the percentage of regenerating fibers was significantly higher when the cross-sectional area was between 500 and 750 μm² (control vs SAV1 KD; p<0.01). In contrast, SAV1 knockdown resulted in a shift toward a larger myofiber size. When the cross-sectional area was >1000 um², the number of myofibers in the SAV1 knockdown group surpassed those in the control group; the percentage was significantly higher when the cross-sectional area ranged between 1750 and 2750 μm².

During skeletal muscle regeneration, vascularization is critical to provide oxygen and nutrients for muscle function. Thus, the inventors stained TA muscle cross-sections with antibodies against the vascular marker CD31 and found a higher number of capillaries per muscle area in the SAV1 KD group than in the control group (698.30±53.40/mm² vs 482.60±18.00/mm², respectively; p<0.05, FIG. 10D, 10E).

Taken together, these findings indicate better muscle regeneration within AAV SAV1 shRNA-treated muscles, further supporting the positive effects of SAV1 knockdown in enhancing myogenesis and angiogenesis.

Aging diminishes the myogenic potential of SCs, resulting in small-caliber myofiber generation after muscle injury or damage. 29 To examine if myogenic SAV1 knockdown promotes SC regenerative capability after muscle injury in aged mice (26-month-old), the inventors used the cardiotoxin-induced mouse muscle injury model followed by AAV9 treatment as described above. The inventors found areas of newly regenerated myofibers with varying sizes on hematoxylin and eosin-stained transverse sections of TA muscles that had been treated with AAV9 for 14 days (FIG. 11A). When the cross-sectional area ranged between 250-500 or 750-1000 um2, the control group had a significantly higher percentage of regenerating myofibers than did the SAV1 knockdown group; when the cross-sectional area ranged between 1750-2750 um², the SAV1 knockdown group had a significantly higher percentage of regenerating myofibers than the control group (FIG. 11B). Therefore, the frequency distribution analysis illustrated higher numbers of regenerative fibers with a large cross-sectional area in the SAV1 knockdown group than in the control group.

At 2 weeks after AAV9 injection, EdU incorporation and Pax7 immunoreactivity were quantified within areas filled with newly regenerated myofibers. A higher percentage of Pax7⁺EdU⁻ SCs (FIG. 11C, white arrows) was found in the SAV1 KD group than in the control group (3.70±0.36% vs 2.15±0.16%, respectively, p<0.05; FIG. 11D). The percentage of EdU⁺ nuclei (4.00±0.26% vs 3.40±0.27%, SAV1 KD vs. control; FIG. 11E) and Pax7⁺EdU⁺ cells (0.23±0.042% vs 0.16±0.060%, SAV1 KD vs. control, FIG. 11F; see yellow arrows in FIG. 11C) remained similar between SAV1 KD and control groups. In addition, neighboring Pax7⁺EdU⁻:Pax7⁻EdU⁺ cells (FIG. 12A, yellow circle) were seen in both AAV9 control and SAV1 shRNA groups. However, Pax7⁺EdU⁺ SC clusters nuclei) were detected only in the AAV9 SAV1 shRNA group (FIG. 12B, yellow arrows). These findings raise the possibility that myogenic SAV1 knockdown promotes muscle regeneration by affecting SC activation and fate determination.

Given that angiogenesis is critical for rebuilding vascular network during muscle regeneration, the inventors used CD31 staining to identify ECs and counted CD31-positive capillaries per muscle area (FIG. 13). The capillary density was significantly higher in the SAV1 KD group than in the control group (642.20±45.43/mm² vs 338.80±9.76/mm², respectively, P<0.05), indicating that after CTX-induced injury, the TA muscle regenerative capability and angiogenesis in aged mice were enhanced in AAV SAV1 shRNA group. Although the formation of new muscle fibers by SCs was apparent, hematoxylin and eosin staining of cardiotoxin-damaged TA muscle also showed histopathologic features of inflammation and the lack of fully restored tissue structures. The inflammatory infiltrates were present in the interstitial spaces (FIG. 14, white arrows) and around myofibers and blood vessels (FIG. 14, yellow and blue arrows).

### Example 6

### Conditioned Medium from Myotubes with Salvador Knockdown Promoted Satellite Cell Proliferation

In addition to myogenic cells, skeletal muscle contains vascular cells, nerve fibers and connective tissues. It is technically challenging to perform in vivo separation of myofiber-derived paracrine factors from those secreted by other cell types that are composed of skeletal muscle in vivo. Therefore, the inventors used two RNA interference methods to target myogenic SAV1 and to examine whether conditioned media collected from Salvador knockdown in myotubes affect SC proliferation in vitro.

First, the inventors performed short interfering RNA (siRNA) transfection with Lipofectamine in C2C12 myotubes. Downregulation of SAV1 mRNA was confirmed by RT-qPCR. Compared to transfection by negative control siRNA (NC siRNA), SAV1 siRNA transfection in myotubes resulted in around 70% knockdown of SAV1 mRNA (1.005±0.198 vs 0.308±0.011, NC siRNA vs SAV1 siRNA, P<0.05; FIG. 15A). Conditioned medium was collected from NC siRNA transfected (NC siRNA-CM) and SAV1 siRNA transfected myotubes (SAV1 siRNA-CM). To assess functional activity of SAV1 siRNA-CM, the inventors maintained SCs either in NC siRNA-CM or in SAV1 siRNA-CM for 24 hours. EdU pulse labeling methods were performed to label SCs that entered the cell cycle. The inventors found a higher percentage of EdU incorporation in cells grown in SAV1 siRNA-CM than in those grown in NC siRNA-CM (FIG. 15B, 15C).

Second, the inventors transduced C2C12 myotubes with AAV9 control and AAV9 SAV1 shRNA (1×10 5 viral genome/cell). The inventors analyzed GFP fluorescence daily to track AAV transduction efficiency (FIG. 15D). Cells were harvested at day 7. Quantification via RT-real time PCR confirmed that the relative expression of SAV1 mRNA was significantly lower in myotubes transduced with AAV9 SAV1 shRNA than in those transduced with AAV9 control (1.001±0.030 vs 0.7336±0.023, AAV9 control vs AAV9 SAV1 shRNA, p<0.05; FIG. 15E). Similar to the above-mentioned approach, when cultured with conditioned medium collected from AAV9 SAV1 shRNA transduced myotubes (AAV SAV1 shRNA-CM), SCs had a higher percentage of EdU labeling as compared to cells cultured with conditioned medium from AAV control transduced myotubes (AAV con-CM; FIG. 15F, 15G).

Together, these results suggest that paracrine factors secreted from SAV1 downregulated myotubes positively regulate SC proliferation.

### Example 7

### Exemplary Methods

### Mouse Models

Unilateral Hindlimb Ischemia

All animal procedures were conducted according to the Texas Heart Institute Animal Welfare Committee guidelines in accordance with the National Institutes of Health Guide for the Care and Use of Laboratory Animals.

To create unilateral hindlimb ischemia, the inventors surgically ligated the left femoral artery in mice (C57BL/6J, 12-15 weeks old; equal numbers of male and female mice; Jackson Laboratory, Bar Harbor, ME) anaesthetized by isoflurane inhalation (2-4% isoflurane in oxygen). Specifically, the inventors placed 2 adjacent sutures on the femoral artery immediately below the branching point of the lateral circumflex femoral artery.¹⁹ Mice were then randomly divided into control and treatment groups; 3 days after surgery, the inventors injected AAV9 control shRNA or AAV9 SAV1 shRNA (1×10¹⁰ viral genomes/mouse) into the gastrocnemius muscle of mice. The inventors monitored the mice and performed the evaluation tests described in the sections below.

Cardiotoxin-Induced Tibialis Anterior Injury

To create a skeletal muscle injury model, the inventors injected cardiotoxin derived from Naja mossambica mossambica (Sigma-Aldrich, St Louis, MO) into the tibialis anterior (TA) muscle in the left legs of adult C57BL/6J mice (10 mol/mouse, 12-15 weeks old; n=2). At 3 days after cardiotoxin injection, mice were euthanized, and the TA muscles were removed and processed. Cardiotoxin-induced damage was confirmed by examining hematoxylin and eosin-stained muscle sections. After successfully creating cardiotoxin-induced TA injury, the inventors conducted the same procedures on cohorts of adult C57BL/6J mice (12-15 weeks old, n=3/group, both sex) and aged mice (26-month-old mice, n=4/group, male, a generous gift from Dr. Darren Woodside). The inventors then randomly divided mice into control and treatment groups; 3 days after cardiotoxin administration, the inventors injected AAV9 control shRNA or AAV9 SAV1 shRNA (1×10¹⁰ viral genomes/mouse) into the damaged TA muscle in the left legs. On day 14 after AAV injection, mice were weighed before TA muscles were removed for obtaining the wet weights. TA muscles were then processed for further analyses.

### AAV9 Vectors

The construction and production of the AAV9 vectors, along with their efficiency in knocking down endogenous SAV1, have been described previously.^{18,20} Briefly, AAV9 GFP (AAV9 control) and the AAV9 SAV1 shRNA viruses were produced by the Intellectual and Developmental Disabilities Research Center Neuroconnectivity Core at Baylor College of Medicine. The inventors used the pENN.AAV.cTNT, p1967-Q vector as the backbone to create AAV9 control or AAV9 SAV shRNA vectors, which contain either a GFP sequence alone or a GFP sequence followed by a miR30-based SAV1 shRNA, under the control of the cardiac troponin T promoter. Either the AAV9 control or AAV9 SAV shRNA vector was co-transfected with pHelper (Addgene 11 2867) and pAAV2/9n (plasmid expressing AAV2 Rep, AAV9 Cap. Addgene 112865) into 293T cells to make the AAV9 control AAV9 SAV shRNA, respectively. At 96-120 hours after transfection, the culture medium and 293T cells were harvested, and viral purification was performed by iodixanol gradient ultracentrifugation.

### Laser Doppler Perfusion Imaging and Treadmill Exhaustion Test

Using a laser Doppler imaging device (Perimed AB, Germany), the inventors measured perfusion before creating ischemia, on day 3 after ischemia (immediately before AAV injection), and every 7 days (weekly) after AAV injection (n=18 mice/group). To minimize temperature-induced blood flow instability, the inventors placed each mouse on a small-animal heated pad at 37° C. for 2 minutes before performing the measurement. Perfusion was expressed as the perfusion ratio in the ischemic leg compared with the contralateral, non-manipulated leg.

At 7 weeks after AAV injection, mice were challenged in a treadmill exhaustion study.21 The belt was set at 6 meters per minute (Eco 3/6, Columbus Instruments, Columbus, OH), and the treadmill velocity was increased 2 meters every 2 minutes and held constant at meters per minute thereafter. Exhaustion was defined as the point at which mice spent >10 consecutive seconds on the shock grid without trying to reengage the treadmill for three times. The inventors recorded the exercise time and distance.

### In Vivo Lectin Labeling of the Vasculature and Sample Processing

The mouse was placed in a restraining device with its tail exposed. Before the lectin injection, the tail was placed in warm water (37° C.) for 5-10 minutes to dilate the vein (n=4 mice/group). Tomato lectin (DyLight 649 labeled from Lycopersicon esculentum, Vector Laboratories, CA) was injected into the tail vein (0.1 mg/mouse). Ten minutes after lectin injection, mice were perfused for 5 minutes with 4% paraformaldehyde in phosphate-buffered saline (PBS) (USB, Cleveland, OH) via the left ventricle. The gastrocnemius muscle was dissected from the leg and fixed in 4% paraformaldehyde at 4° C. overnight. Tissue samples were transferred to 15% sucrose in PBS for 2 hours followed by incubation in 30% sucrose in PBS at 4° C. overnight. The samples were embedded in Tissue-Tek OCT compound (Sakura, Torrance, CA) in dry ice and cut on a cryostat (Leica CM1950). Muscle cryosections (10 μm) were fixed with acetone for 5 minutes and washed, and nuclei were counterstained with DAPI (Vector Laboratories). Fluorescence images were obtained with a confocal laser scanning microscope (Leica TCS SP5II, Buffalo Grove, IL). To quantify lectin-positive capillary numbers per myofiber for each mouse muscle sample, the inventors sampled three sections from each mouse. The inventors counted at least 150 randomly selected myofibers from one muscle cryosection at the center of the AAV9 injection site, one from the proximal region, and one from the distal region of injection site. The average was used to represent each mouse for statistical analyses.

### Western Blot Analysis

Gastrocnemius muscles were lysed in an ice-cold lysis buffer (10 mM Tris-HCl, pH 7.6, 3 mM MgCl₂, 40 mM KCl, 2 mM DTT, 5% glycerol, 0.5% NP40) containing a protease inhibitor cocktail (Roche, Basal, Switzerland) on ice. Lysates were sonicated (Bioruptor 300, Diagenode, Belgium) and centrifuged at 12,000 g at 4° C. for 10 minutes. Supernatants were collected, and the proteins were quantified (Bio-Rad DC Protein Assay Reagents, Hercules, CA). A total 30 μg of protein from each sample was fractionated by SDS-PAGE (4-20% gradient gel, Bio-Rad) and transferred onto Immun-Blot polyvinylidene fluoride membranes (Bio-Rad), which were incubated in a TBS-Tween solution containing 5% non-fat dry milk for 1 hour at room temperature. The blots were then incubated overnight at 4° C. with mouse anti-SAV1 antibody (sc-101205, Santa Cruz Biotechnology, Dallas, TX) mouse anti-YAP antibody (sc-101199, Santa Cruz Biotechnology), and rabbit anti-Phospho-YAP (Ser127, 4911s, Cell Signaling, Danvers, MA), respectively, followed by incubation with horseradish peroxidase (HRP)-conjugated goat anti-mouse Kappa or HRP-conjugated goat anti-rabbit IgG (both secondary antibodies, SouthernBiotech, Birmingham, AL) for 45 minutes at room temperature.

Protein signals were detected by using the enhanced chemiluminescence system (Thermo Fisher Scientific, Waltham, MA). To verify equal loading of each protein sample, the inventors stripped and reprobed the membranes with HRP-conjugated goat antibody for GAPDH (sc-20357, Santa Cruz Biotechnology). Average protein expression levels were measured by using a densitometric program in NIH ImageJ software.

Because of mouse-to-mouse variability in SAV1 and pYAP protein expression at basal conditions (FIG. 4A, see protein expression variation on non-ischemic contralateral legs of mouse 1-mouse 4), the inventors performed a semi-quantitative analysis according to the following procedures. First, the inventors performed normalization experiments by using an internal control GAPDH to adjust for loading variation. The inventors then determined the normalized SAV1, total YAP, and pYAP protein response to ischemia by measuring the fold change of their expression in ischemic legs relative to contralateral legs (FIG. 4).

For semi-qualification of the western blot in FIG. 5, after performing normalization procedures as described above, the inventors determined the normalized SAV1 and pYAP protein response to either AAV9 con shRNA or AAV9 SAV1 shRNA treatment by measuring their expression in AAV9-treated ischemic legs relative to contralateral legs. Finally, the relative protein levels in AAV9 SAV1 shRNA treated ischemic legs were expressed as fold changes in AAV9 SAV1 shRNA-treated ischemic legs versus AAV control shRNA-treated ischemic legs.

### Immunofluorescence and Histological Analysis

To label cell proliferation in vivo, the inventors injected 5-ethynyl-2′-deoxyuridine (EdU, 500 μg/mouse, Thermo Fisher Scientific) intraperitoneally into mice both on day 3 and at 4 hours before euthanasia. After euthanasia, the gastrocnemius and TA muscles were harvested and processed by using published methods. 22 Muscle cryosections (10m) were fixed with acetone for 5 minutes and treated with 0.2% triton x-100 in PBS for 15 minutes. EdU staining was performed according to manufacturer's instructions (Click-iT EdU cell proliferation kit for imaging, Alexa Fluor 647 dye, Thermo Fisher Scientific). The muscle slides were incubated with the following primary antibodies individually at 4° C. overnight: rabbit anti-GFP (ab290, Abcam, Cambridge, United Kingdom); rabbit anti-laminin (L9393, Sigma-Aldrich); rabbit anti-YAP1 (NB110-58358, Novus Biologicals, Littleton, CO); mouse anti-Pax7 (DSHB); and rabbit anti-CD31 (ab 28364, Abcam), respectively. The sections were then incubated with the corresponding secondary antibodies: Alexa Fluor-488 donkey anti-rabbit IgG, Alexa Fluor-555 donkey anti-rabbit IgG, and Alexa Fluor-555 donkey anti-mouse IgG (all from Thermo Fisher Scientific). Nuclei were counterstained with DAPI (Vector Laboratories). Fluorescence images of stained sections were taken with a confocal laser scanning microscope (Leica TCS SP5II, Buffalo Grove, IL). Image processing and quantitative analysis were performed by using ImageJ software. For each mouse tissue sample, the inventors selected a total of three sections for quantification, including one muscle transverse section from the center of the AAV9 injection site, one from the proximal region, and one from the distal region. The average was used to represent each mouse for statistical analyses.

As described previously,²² TA muscle slides (6-μm) were air-dried at room temperature, stained with hematoxylin and eosin (Sigma-Aldrich), and then mounted with a permanent mounting medium (Vector Laboratories). The inventors used an Olympus microscope (BX-51) to obtain the images. ImageJ software was used to measure the cross-sectional area of each regenerating myofiber. A total range of 500-600 fibers in each group was measured.

### C2C12 Myotube Conditioned Medium Preparation

siRNA Knockdown of SAV1

Mouse C2C12 myoblasts (ATCC, Manassas, VA, USA) were cultured in 6-well tissue culture plates with high glucose Dulbecco's Modified Eagle's medium (DMEM) containing 10% fetal bovine serum (FBS) and 1% penicillin-streptomycin (PS) in a 5% CO2 incubator at 37° C. To induce C2C12 myoblast differentiation to myotubes, the inventors replaced the medium with DMEM supplemented with 2% horse serum and 1% PS in a 5% CO2 incubator at 37° C. On day 4 of C2C12 myoblast differentiation into myotubes, silencer select SAV1 siRNA (assay ID 182232)-Lipofectamin RNAiMAX complexes or silencer negative control siRNA (#AM4611)-Lipofectamin RNAiMAX complexes were transfected into myotubes according to the manufacturer's procedures (Thermo Fisher Scientific). At 48 hours after transfection, the supernatants were aspirated. The myotubes were washed once with basic DMEM supplemented with 0.5% FBS and 1% PS and were maintained in basic DMEM supplemented with 0.5% FBS and 1% PS in a 5% CO2 incubator at 37° C. for another 24 hours. Cell culture supernatants (conditioned medium stock solution) were collected, centrifuged at 2000 rpm for 5 minutes, aliquoted, and stored at −80° C.

AAV9 Mediated Knockdown of SAV1

On day 2 of myoblast differentiation, cells in 6-well plates were washed once with serum-free DMEM followed by incubation in serum-free DMEM containing either AAV9 control shRNA or AAV9 SAV1 shRNA (1×105 viral genome/cell) in a 5% CO2 incubator at 37° C. After 1 hour, 100 multiplicities of infection (moi) of wild-type adenovirus (MD Anderson Cancer Center Vector Core) were added to AAV9-infected cells to enhance AAV9 transduction efficiency. At one hour after adenovirus infection, 2 mL of DMEM supplemented with 2% horse serum and 1% PS was added to each well, and myotubes were incubated for 6 days. The culture supernatants were aspirated, and myotubes were washed once with DMEM supplemented with 0.5% FBS and 1% PS before being cultured in the same medium in a 5% CO2 incubator at 37° C. for another 24 hours. Cell culture supernatants (conditioned medium stock solution) were collected, centrifuged at 2000 rpm for 5 minutes. The supernatant was aliquoted and stored at −80° C. Live cell images were taken with an Olympus Ix71 fluorescence inverted fluorescence & phase contrast tissue culture microscope.

RT-Real Time PCR

After the treatments described above, myotubes were washed with cold Dulbecco's phosphate-buffered saline and collected for RNA isolation (RNase Plus Micro Kit, Qiagen). Total RNA (2 μg) was reverse transcribed using high-capacity RNA-to-cDNA kit (Invitrogen) and T100 thermal cycler (Bio-Rad, Hercules, CA, USA). qPCR was performed using TaqMan Gene Expression Master Mix (Invitrogen) and QuantStudio 6 Real-Time PCR System (Life Technologies, Grand Island, NY, USA). SAV1-specific primers/probes (assay ID Mm01292174 ml) and 18S rRNA endogenous control (VIC/MGB Probe) were purchased from Thermo Fisher Scientific. The relative expression of RNA was calculated using QuantStudio Real-Time PCR software (the ΔΔCt method). All experiments were performed in triplicate in three independent experiments.

Satellite Cell Isolation, Culturing, and Labeling with EdU

After mice were euthanized, the gastrocnemius and TA muscles were harvested, minced, and digested with collagenase type II (Worthington, Lakewood, NJ). Cells were dissociated from digested tissue fragments by using published methods. 23 The cell suspension was passed through a 40-μm cell strainer, and red blood cells were lysed by using 1x lysing buffer (BD Bioscience). Cells were washed and resuspended with PBS containing 0.5% BSA. The cell suspension was incubated with antibody cocktail from the mouse SC isolation kit (#130-104-268, Miltenyi Biotec) for 20 minutes at 4° C. Cells were washed, resuspended, and passed through a 30-μm pre-separation filter to a LS column (Miltenyi Biotec) sitting on a magnetic separator. The LS column was then washed 3 times with PBS containing 0.5% BSA. All flow-through fractions were combined and centrifuged at 1500 rpm at for 5 minutes.

The cell pellet was resuspended and counted, and SCs were cultured in growth medium (DMEM containing 20% FBS, 1% chick embryo extract, and 1% PS) until they reached 80% confluence in the culture dishes (60×15 mm). Cells were sub-cultured into 4-well Nunc Lab Tek chamber slides (Nalge Nunc International, Naperville, IL) for 24 hours. The cell culture supernatant was aspirated, and cells were washed once with basic DMEM and incubated with fresh conditioned medium (generated from 40% of conditioned medium stock solution mixing with 60% of growth medium, v/v) for 24 hours in a 5% CO2 incubator at 37° C. EdU was added to the culture media (final concentration, 10 μM).

After 4 hours, the culture supernatant was aspirated, and the cells were washed 2 times with PBS before being fixed with 4% paraformaldehyde. EdU staining of cells was performed according to the manufacturer's instructions (Click-iT EdU cell proliferation kit for imaging, Alexa Fluor 488 dye, Thermo Fisher Scientific). Fluorescence images were taken with a confocal laser scanning microscope (Leica TCS SP5II, Buffalo Grove, IL) and with an Olympus Ix71 fluorescence inverted fluorescence & phase contrast tissue culture microscope. Quantitative analyses were performed by using ImageJ software. All experiments were performed in triplicate in five independent experiments.

### Statistical Analysis

Data were expressed as mean±standard error of mean (SEM). The nonparametric Mann Whitney test or an unpaired t test was used to determine statistical significance between two groups at the same time point. For the western blot analysis of more than 2 groups (FIG. 5C, 5D), the inventors used a one-way ANOVA with Dunnett's multiple comparison test (Graph Pad Prism 7). P<0.05 was considered statistically significant.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

1 Hirsch A T, Duval S. The global pandemic of peripheral artery disease. Lancet. 2013; 382(9901):1312-1314.
2 Hammer A, Steiner S. Gene therapy for therapeutic angiogenesis in peripheral arterial disease—a systematic review and meta-analysis of randomized, controlled trials. Vasa. 2013; 42(5):331-339.
3 Frangogiannis N G. Cell therapy for peripheral artery disease. Curr Opin Pharmacol. 2018; 39:27-34.
4 Inampudi C, Akintoye E, Ando T, et al. Angiogenesis in peripheral arterial disease. Curr Opin Pharmacol. 2018; 39:60-67.
5 Pipinos, II, Judge A R, Selsby J T, et al. The myopathy of peripheral arterial occlusive disease: part 1. Functional and histomorphological changes and evidence for mitochondrial dysfunction. Vasc Endovascular Surg. 2007; 41(6):481-489.
6 McDermott M M. Lower extremity manifestations of peripheral artery disease: the pathophysiologic and functional implications of leg ischemia. Circ Res. 2015; 116(9):1540-1550.
7 Brack A S, Conboy M J, Roy S, et al. Increased Wnt signaling during aging alters muscle stem cell fate and increases fibrosis. Science. 2007; 317(5839):807-810.
8 Almada A E, Wagers A J. Molecular circuitry of stem cell fate in skeletal muscle regeneration, ageing and disease. Nat Rev Mol Cell Biol. 2016; 17(5):267-279.
9 Wosczyna M N, Rando T A. A muscle stem cell support group: Coordinated cellular responses in muscle regeneration. Dev Cell. 2018; 46(2):135-143.
10 Charge S B, Rudnicki M A. Cellular and molecular regulation of muscle regeneration. Physiol Rev. 2004; 84(1):209-238.
11 Montarras D, Morgan J, Collins C, et al. Direct isolation of satellite cells for skeletal muscle regeneration. Science. 2005; 309(5743):2064-2067.
12 Yin H, Price F, Rudnicki M A. Satellite cells and the muscle stem cell niche. Physiol Rev. 2013; 93(1):23-67.
13 Bentzinger C F, Wang Y X, Dumont N A, et al. Cellular dynamics in the muscle satellite cell niche. EMBO Rep. 2013; 14(12):1062-1072.
14 Snijders T, Nederveen J P, McKay B R, et al. Satellite cells in human skeletal muscle plasticity. Front Physiol. 2015; 6:283.
15 Christov C, Chretien F, Abou-Khalil R, et al. Muscle satellite cells and endothelial cells: close neighbors and privileged partners. Mol Biol Cell. 2007; 18(4):1397-1409.
16 Wang Y, Yu A, Yu F X. The Hippo pathway in tissue homeostasis and regeneration. Protein Cell. 2017; 8(5):349-359.
17 Zhang L, Yue T, Jiang J. Hippo signaling pathway and organ size control. Fly (Austin). 2009; 3(1):68-73.
18 Leach J P, Heallen T, Zhang M, et al. Hippo pathway deficiency reverses systolic heart failure after infarction. Nature. 2017; 550(7675):260-264.
19 Kochi T, Imai Y, Takeda A, et al. Characterization of the arterial anatomy of the murine hindlimb: functional role in the design and understanding of ischemia models. PloS One. 2013; 8(12):e84047.
20 Morikawa Y, Heallen T, Leach J, et al. Dystrophin-glycoprotein complex sequesters Yap to inhibit cardiomyocyte proliferation. Nature. 2017; 547(7662):227-231.
21 Deng Y, Yang Z, Terry T, et al. Prostacyclin-producing human mesenchymal cells target H19 lncRNA to augment endogenous progenitor function in hindlimb ischaemia. Nat Commun. 2016; 7:11276.
22 Guardiola O, Andolfi G, Tirone M, et al. Induction of acute skeletal muscle regeneration by cardiotoxin injection. J Vis Exp. 2017(119).
23 Motohashi N, Asakura Y, Asakura A. Isolation, culture, and transplantation of muscle satellite cells [in eng]. J Vis Exp. JoVE 2014(86).
24. Liu S, Martin J F. The regulation and function of the Hippo pathway in heart regeneration. Wiley Interdiscip Rev Dev Biol. 2019; 8(1):e335.
25. von Maltzahn J, Jones A E, Parks R J et al. Pax7 is critical for the normal function of satellite cells in adult skeletal muscle [in eng]. Proc. Natl Acad. Sci. U.S.A. 2013; 110(41):16474-16479.
26 Lepper C, Partridge T A, Fan C M. An absolute requirement for Pax7-positive satellite cells in acute injury-induced skeletal muscle regeneration [in eng]. Development (Cambridge, England) 2011; 138(17):3639-3646.
27 van der Ven P F, Schaart G, Jap P H et al. Differentiation of human skeletal muscle cells in culture: maturation as indicated by titin and desmin striation [in eng]. Cell Tissue Res. 1992; 270(1):189-198.
28 Vaittinen S, Lukka R, Sahlgren C et al. The expression of intermediate filament protein nestin as related to vimentin and desmin in regenerating skeletal muscle [in eng]. J. Neuropathol. Exp. Neurol. 2001; 60(6):588-597.
29 Conboy I M, Conboy M J, Wagers A J, et al. Rejuvenation of aged progenitor cells by exposure to a young systemic environment. Nature. 2005; 433(7027):760-764.
30 Bobadilla M, Sainz N, Abizanda G et al. The CXCR4/SDF1 axis improves muscle regeneration through MMP-10 activity [in eng]. Stem Cells Devel. 2014; 23(12):1417-1427.
31 Heallen T, Zhang M, Wang J, et al. Hippo pathway inhibits Wnt signaling to restrain cardiomyocyte proliferation and heart size. Science. 2011; 332(6028):458-461.
32 Harvey K, Tapon N. The Salvador-Warts-Hippo pathway—an emerging tumour-suppressor network. Nat Rev Cancer. 2007; 7(3):182-191.
33 Pan D. The hippo signaling pathway in development and cancer. Dev Cell. 2010; 19(4):491-505.
34 Lu L, Li Y, Kim S M, et al. Hippo signaling is a potent in vivo growth and tumor suppressor pathway in the mammalian liver. Proc Natl Acad Sci USA. 2010; 107(4):1437-1442.
35 Watt K I, Harvey K F, Gregorevic P. Regulation of tissue growth by the mammalian Hippo signaling pathway. Front Physiol. 2017; 8:942.
36 Yang Z, Nakagawa K, Sarkar A, et al. Screening with a novel cell-based assay for TAZ activators identifies a compound that enhances myogenesis in C2C12 cells and facilitates muscle repair in a muscle injury model. Mol Cell Biol. 2014; 34(9):1607-1621.
37 Watt K I, Turner B J, Hagg A, et al. The Hippo pathway effector YAP is a critical regulator of skeletal muscle fibre size. Nat Commun. 2015; 6:6048.
38 Goodman C A, Dietz J M, Jacobs B L, et al. Yes-Associated Protein is up-regulated by mechanical overload and is sufficient to induce skeletal muscle hypertrophy. FEBS Lett. 2015; 589(13):1491-1497.
39 Wei B, Dui W, Liu D, et al. MST1, a key player, in enhancing fast skeletal muscle atrophy. BMC Biol. 2013; 11:12.
40 Forster R, Liew A, Bhattacharya V, et al. Gene therapy for peripheral arterial disease. Cochrane Database Syst Rev. 2018; 10:Cd012058.
41 Hardee C L, Arevalo-Soliz L M, Hornstein B D, et al. Advances in non-viral DNA vectors for gene therapy. Genes (Basel). 2017; 8(2).
42 Gorenoi V, Brehm M U, Koch A, et al. Growth factors for angiogenesis in peripheral arterial disease. Cochrane Database Syst Rev. 2017; 6:Cd011741.
43 Sanada F, Taniyama Y, Muratsu J, et al. Gene-therapeutic strategies targeting angiogenesis in peripheral artery disease. Medicines (Basel). 2018; 5(2).
44 Wang D, Tai P W L, Gao G. Adeno-associated virus vector as a platform for gene therapy delivery. Nat Rev Drug Discov. 2019; 18(5):358-378.
45 Pattali R, Mou Y, Li X J. AAV9 Vector: a Novel modality in gene therapy for spinal muscular atrophy. Gene Ther. 2019; 26(7-8):287-295.
46 Al-Zaidy S A, Mendell J R. From clinical trials to clinical practice: Practical considerations for gene replacement therapy in SMA Type 1. Pediatr Neurol. 2019; 100:3-11.
47 McHugh D, Gil J. Senescence and aging: Causes, consequences, and therapeutic avenues. J Cell Biol. 2018; 217(1):65-77.
48 Xie Q, Chen J, Feng H, et al. YAP/TEAD-mediated transcription controls cellular senescence. Cancer Res. 2013; 73(12):3615-3624.
49 Shao D, Zhai P, Del Re D P, et al. A functional interaction between Hippo-YAP signalling and FoxO1 mediates the oxidative stress response. Nat Commun. 2014; 5:3315-3315.
50 O'Keefe E, siRNAs and shRNAs: Tools for Protein Knockdown by Gene Silencing. Mater. Methods 2013; 3:197.
51 Chery J, RNA therapeutics: RNAi and antisense mechanisms in clinical applications. Postdoc J. 2016: 4(7):35-50.

What is claimed is:

1. A method of increasing angiogenesis in skeletal muscle, the method comprising delivering to skeletal muscle cells an effective amount of a composition comprising at least one inhibitory nucleic acid, wherein the inhibitory nucleic acid targets Salvador.

2. A method of regenerating myofibers in skeletal muscle, the method comprising delivering to skeletal muscle cells an effective amount of a composition comprising at least one inhibitory nucleic acid, wherein the inhibitory nucleic acid targets Salvador.

3. A method of inducing proliferation of satellite cells in skeletal muscle, the method comprising delivering to the satellite cells an effective amount of a composition comprising at least one inhibitory nucleic acid, wherein the inhibitory nucleic acid targets Salvador.

4. A method of treating limb ischemia in a mammalian subject, the method comprising delivering to skeletal muscle cells of an ischemic limb in the subject an effective amount of a composition comprising at least one inhibitory nucleic acid, wherein the inhibitory nucleic acid targets Salvador.

5. The method of any preceding claim, wherein the inhibitory nucleic acid has, or is encoded by a sequence having, at least 80% identity to a nucleotide sequence selected from the group consisting of SEQ ID NO: 2, SEQ ID NO: 3, and SEQ ID NO: 4.

6. The method of any preceding claim, wherein the composition comprises (i) an inhibitory nucleic acid having, or encoded by a sequence having, at least 80% identity to SEQ ID NO: 2, (ii) an inhibitory nucleic acid having, or encoded by a sequence having, at least 80% identity to SEQ ID NO: 3, and (iii) an inhibitory nucleic acid having, or encoded by a sequence having, at least 80% identity to SEQ ID NO: 4.

7. The method of any preceding claim, wherein the inhibitory nucleic acid has, or is encoded by a sequence having, at least 85%, 90%, 95%, 96%, 97%, 98%, or 99% identity to a sequence selected from the group consisting of SEQ ID NO: 2, SEQ ID NO: 3, and SEQ ID NO: 4.

8. The method of any preceding claim, wherein the inhibitory nucleic acid has a sequence, or is encoded by a sequence, selected from the group consisting of SEQ ID NO: 2, SEQ ID NO: 3, and SEQ ID NO: 4.

9. The method of any one of claims 1 to 8, wherein the inhibitory nucleic acid is an antisense DNA molecule.

10. The method of any one of claims 1 to 8, wherein the inhibitory nucleic acid is an RNA.

11. The method of claim 10, wherein the inhibitory nucleic acid is a short hairpin RNA (shRNA).

12. The method of claim 11, wherein the shRNA is at least 43 nucleotides in length.

13. The method of claim 11, wherein the shRNA is less 138 nucleotides in length.

14. The method of claim 11, wherein the shRNA comprises a loop structure of between 5 and 19 nucleotides in length.

15. The method of any one of claims 10 to 14, wherein a nucleotide sequence encoding the RNA is comprised in a nucleic acid construct, and wherein the RNA is expressed in the skeletal muscle cells.

16. The method of claim 15, wherein the nucleotide sequence encoding the RNA is operably linked to a tissue-specific promoter.

17. The method of claim 16, wherein the promoter is a cardiac troponin T promoter.

18. The method of claim 15, wherein the nucleic acid construct comprises a post-transcriptional regulatory element.

19. The method of claim 18, wherein the post-transcriptional regulatory element is a woodchuck hepatitis virus post-transcriptional regulatory element (WPRE).

20. The method of any preceding claim, wherein a nucleotide sequence encoding the inhibitory nucleic acid is comprised in a vector.

21. The method of claim 20, wherein the vector is a non-viral vector.

22. The method of claim 20, wherein the vector is a non-integrating vector.

23. The method of claim 20, wherein the vector is viral vector.

24. The method of claim 23, wherein the vector is an adeno-associated viral (AAV) vector.

25. The method of claim 23, wherein the vector is a lentiviral vector.

26. The method of claim 6, wherein nucleotide sequences encoding the inhibitory nucleic acids are comprised in a single nucleic acid construct, and wherein the nucleotide sequences are expressed in the skeletal muscle cells or satellite cells.

27. The method of claim 26, wherein the nucleotide sequences encoding the inhibitory nucleic acids are regulated by a single promoter.

28. The method of any one of claims 1 to 4, wherein the composition comprises a nucleic acid construct comprising: (i) a nucleic acid having the nucleotide sequence set forth in SEQ ID NO: 2, (ii) a nucleic acid having the nucleotide sequence set forth in SEQ ID NO: 3, and (iii) a nucleic acid having the nucleotide sequence set forth in SEQ ID NO: 4; wherein nucleic acids (i)-(iii) are operably linked to a promoter.

29. The method of claim 28, wherein the promoter is a cardiac troponin T promoter.

30. The method of claim 28 or 29, wherein the nucleic acid construct comprises sequences encoding a 3′ microRNA-30 sequence and a 5′ microRNA-30 sequence.

31. The method of any one of claims 28 to 30, wherein the nucleic acid construct is comprised in a viral vector.

32. The method of claim 31, wherein the vector is an adeno-associated viral (AAV) vector.

33. The method of claim 31, wherein the vector is a lentiviral vector.

34. The method of claim 31, wherein the nucleic acid construct comprises a post-transcriptional regulatory element.

35. The method of claim 34, wherein the post-transcriptional regulatory element is a woodchuck hepatitis virus post-transcriptional regulatory element (WPRE).

36. The method of any one of claims 31 to 35, wherein the nucleic acid construct comprises 5′ and 3′ inverted terminal repeats.

37. The method of any preceding claim, wherein the method is an in vitro or ex vivo method.

38. The method of any one of claims 1 to 36, wherein the composition is administered to a mammalian subject.

39. The method of claim 38, wherein the skeletal muscle is ischemic.

40. The method of claim 38, wherein the skeletal muscle is atrophied.

41. The method of claim 38, wherein the skeletal muscle has suffered traumatic injury.

42. The method of claim 38, wherein the mammalian subject has a condition selected from the group consisting of limb ischemia, peripheral vascular disease, and sarcopenia.

43. An inhibitory nucleic acid for use in a method of increasing angiogenesis in skeletal muscle, the method comprising delivering to the skeletal muscle an effective amount of a composition comprising at least one inhibitory nucleic acid that targets Salvador.

44. An inhibitory nucleic acid for use in a method of regenerating myofibers in skeletal muscle, the method comprising delivering to the skeletal muscle an effective amount of a composition comprising at least one inhibitory nucleic acid that targets Salvador.

45. An inhibitory nucleic acid for use in a method of inducing proliferation of satellite cells in skeletal muscle, the method comprising delivering to the satellite cells an effective amount of a composition comprising at least one inhibitory nucleic acid that targets Salvador.

46. An inhibitory nucleic acid for use in a method of treating limb ischemia in a mammalian subject, the method comprising delivering to skeletal muscle of an ischemic limb in the subject an effective amount of a composition comprising at least one inhibitory nucleic acid, wherein the inhibitory nucleic acid targets Salvador.
